SEQUENCE LISTING (27 sequences)

SEQ ID NO: 1 - protein, 530 aa, Chandipura virus
MTSSVTISVILLISFIAPSYSSLSIAFPENTKLDWKPVTKNTRYCPMGGEWFLEPGLQEESFLSSTPIGATPSKSDGFLCHAAKWVTTCDFRWYGPKYITHSIHNIKPTRSDCDTALASYKSGTLVSPGFPPESCGYASVTDSEFLVIMITPHHVGVDDYRGHWVDPLFVGGECDQSYCDTIHNSSVWIPADQTKKNICGQSFTPLTVTVAYDKTKEIAAGAIVFKSKYHSHMEGARTCRLSYCGRNGIKFPNGEWVSLDVKTKIQEKPLLPLFKECPAGTEVRSTLQSDGAQVLTSEIQRILDYSLCQNTWDKVERKEPLSPLDLSYLASKSPGKGLAYTVINGTLSFAHTRYVRMWIDGPVLKEMKGKRESPSGISSDIWTQWFKYGDMEIGPNGLLKTAGGYKFPWHLIGMGIVDNELHELSEANPLDHPQLPHAQSIADDSEEIFFGDTGVSKNPVELVTGWFTSWKESLAAGVVLILVVVLIYGVLRCFPVLCTTCRKPKWKKGVERSDSFEMRIFKPNNMRARV

SEQ ID NO: 2 - DNA, 1592 bp, Chandipura virus
  source 1..1592: mol_type = genomic DNA
atgacttcttcagtgacaattagtgtgatccttcttatctcctttattgccccatcatactcatctttgagtatagcatttccagaaaacaccaaattagattggaagccagtcacaaaaaacactagatactgccctatgggtggggaatggtttctagaaccagggttacaagaagaatctttcttgagctctacacccattggtgcgaccccctccaagtcagatggatttctctgtcatgcagccaagtgggtgacaacatgtgatttcagatggtatggacccaaatatattacgcattcaattcataatatcaaacctacccgatcagattgtgatacagcgcttgcatcatacaaatccgggacattagtgagccctggttttcccccagagtcttgtggttatgcttctgtgactgactccgagttcctggtgatcatgattacccctcatcacgtgggtgtggatgactacagaggacattgggtagatcctctttttgttggaggagaatgcgaccagtcttattgtgatactatccacaactcctcagtttggattcctgctgatcagactaagaagaacatttgcggccagtcctttaccccactgactgtgacggtgcttatgataaaaccaaagaaattgctgcaggcgcaatagtctttaagagcaaatatcactctcacatggaaggtgctcgaacttgcagattgagttattgcggtcggaacggaattaaattccccaatggagagtgggtcagcctggatgttaaaactaagatccaagagaaacctttacttcccttgtttaaagagtgtcctgctgggacagaggtgagatctactcttcaatccgatggggctcaagtcttgacctcggagattcagaggattttggattattccttgtgtcagaacacgtgggacaaggtagaacgcaaagagcctttgtctccattggatctcagctatttggcatctaaatccccggggaaaggtctggcatatacagtgataaatgggacattgtcatttgcccataccagatacgtgaggatgtggattgatggcccggtgttgaaagaaatgaaaggcaaaagggaatctcctagtgggatctcgagtgatatttggacccaatggttcaaatatggggatatggagataggcccaaacggcctcttaaagacagcaggagggtacaaattcccctggcatctgatcggtatgggaattgtggacaatgaactacacgagctcagtgaggcaaaccctttagaccatccacagctacctcatgctcagtctattgccgacgattcggaggagatcttctttggagacactggggtttccaagaatccagtagaactagttacagggtggttcactagctggaaagagagcttagctgccggtgttgttttgatattggtagttgtcctgatttatggtgtcctccgttgtttcccggtgttgtgtactacctgcagaaagcccaaatggaagaaaggggtagagaggtccgatagctttgagatgcggattttcaagcccaacaacatgagagccagagtatga

SEQ ID NO: 3 - protein, 535 aa, Chandipura virus
MTSSVTISVILLISFIAPSYSSLSIAFPENTKLDWKPVTKNTRYCPMGGEWFLEPGLQEESFLSSTPIGATPSKSDGFLCHAAKWVTTCDFRWYGPKYITHSIHNIKPTRSDCDTALASYKSGTLVSPGFPPESCGYASVTDSEFLVIMITPHHVGVDDYRGHWVDPLFVGGECDQSYCDTIHNSSVWIPADQTKKELDWKANICGQSFTPLTVTVAYDKTKEIAAGAIVFKSKYHSHMEGARTCRLSYCGRNGIKFPNGEWVSLDVKTKIQEKPLLPLFKECPAGTEVRSTLQSDGAQLTSEIQRILDYSLCQNTWDKVERKEPLSPLDLSYLASKSPGKGLAYTVINGTLSFAHTRYVRMWIDGPVLKEMKGKRESPSGISSDIWTQWFKYGDMEIGPNGLLKTAGGYKFPWHLIGMGIVDNELHELSEANPLDHPQLPHAQSIADDSEEIFFGDTGVSKNPVELVTGWFTSWKESLAAGVVLILVVVLIYGVLRCFPVLCTTCRKPKWKKGVERSDSFEMRIFKPNNMRARV

SEQ ID NO: 4 - protein, 523 aa, Carajas virus
MKMKMVIAGLILCIGILPAIGKITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 5 - protein, 519 aa, Carajas virus
MKMKMVIAGLILCIGILPAIGKITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEMADKDLFAAARFPECPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 6 - DNA, 1572 bp, Carajas virus
  source 1..1572: mol_type = genomic DNA
atgaagatgaaaatggtcatagcaggattaatcctttgtatagggattttaccggctattgggaaaataacaatttctttcccacaaagcttgaaaggagattggaggcctgtacctaagggatacaattattgtcctacaagtgcggataaaaatctccatggtgatttgattgacataggtctcagacttcgggcccctaagagcttcaaagggatctccgcagatggatggatgtgccatgcggcaagatggatcaccacctgtgatttcagatggtatggacccaagtacatcacccactcaattcactctttcaggccgagcaatgaccaatgcaaagaagcaatccggctgactaatgaagggaattggattaatccaggtttccctccgcaatcttgcggatatgcttctgtaaccgactcagaatccgttgtcgtaaccgtgaccaagcaccaggtcctagtagatgagtactccggctcatggatcgatagtcaattccccggaggaagttgcacatcccccatttgcgatacagtgcacaactcgacactttggcacgcggaccacaccctggacagtatctgtgaccaagaattcgtggcaatggacgcagttctgttcacagagagtggcaaatttgaagagttcggaaaaccgaactccggcatcaggagcaactattttccttatgagagtctgaaagatgtatgtcagatggatttctgcaagaggaaaggattcaagctcccatccggtgtctggtttgaaatcgaggatgcagagaaatctcacaaggcccaggttgaattgaaaataaaacggtgccctcatggagcagtaatctcagctcctaatcagaatgcagcagatatcaatctgatcatggatgtggaacgaattctagactactccctttgccaagcaacttggagcaaaatccaaaacaaggaagcgttgacccccatcgatatcagttatcttggtccgaaaaacccaggaccaggcccagccttcaccataataaatggaacactgcactacttcaatactagatacattcgagtggatattgcagggcctgttaccaaagagattacaggatttgtttcgggaacatctacatctagggtgctgtgggatcagtggttcccatatggagagaattccattggacccaatggcttgctgaaaaccgccagcggatacaaatatccattgttcatggttggtacaggtgtgctggatgcggacatccacaagctgggagaagcaaccgtgattgaacatccacatgccaaagaggctcagaaggtagttgatgacagtgaggttatattttttggtgacaccggagtctccaagaatccagtggaggtagtcgaaggatggtttagcggatggagaagctctttgatgagcatatttggcataattttgttgattgtttgtttagtcttgattgttcgaatccttatagcccttaaatactgttgtgttagacacaaaaagagaactatttacaaagaggaccttgaaatgggtcgaattcctcggagggcttaa

SEQ ID NO: 7 - protein, 1254 aa, SARS-CoV-2 virus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 8 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 9 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 10 - protein, 517 aa, Vesicular stomatitis virus
MLSYLIFALVVSPILGKIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYKDGVSFNPGFPPQSCGYGTVTDAEAHIVTVTPHSVKVDEYTGEWIDPHFIGGRCKGQICETVHNSTKWFTSSDGESVCSQLFTLVGGTFFSDSEEITSMGLPETGIRSNYFPYVSTEGICKMPFCRKPGYKLKNDLWFQITDPDLDKTVRDLPHIKDCDLSSSIVTPGEHATDISLISDVERILDYALCQNTWSKIEAGEPITPVDLSYLGPKNPGAGPVFTIINGSLHYFMSKYLRVELESPVIPRMEGKVAGTRIVRQLWDQWFPFGEVEIGPNGVLKTKQGYKFPLHIIGTGEVDNDIKMERIVKHWEHPHIEAAQTFLKKDDTEEVLYYGDTGVSKNPVELVEGWFSGWRSSIMGVLAVIIGFVILIFLIRLIGVLSSLFRQKRRPIYKSDVEMAHFR

SEQ ID NO: 11 - protein, 1554 aa, Vesicular stomatitis virus
ATGTTGTCTTATCTAATCTTTGCACTTGTCGTTTCGCCCATTTTGGGTAAAATAGAAATCGTCTTCCCTCAACACACCACTGGAGACTGGAAGAGAGTTCCCCATGAATATAACTATTGCCCTACCAGCGCAGACAAGAATTCACATGGGACTCAAACAGGAATCCCTGTTGAGTTGACAATGCCCAAAGGACTGACAACCCATCAAGTGGATGGATTTATGTGTCACTCAGCGTTGTGGATGACCACTTGTGACTTCAGATGGTATGGACCAAAATATATAACCCATTCCATACATAATGAGGAGCCTACAGATTATCAATGTTTGGAGGCCATCAAGGCATACAAAGATGGAGTCAGTTTCAACCCAGGTTTTCCTCCTCAGAGCTGTGGATATGGGACAGTTACAGATGCTGAGGCCCATATTGTGACAGTTACTCCCCACTCTGTCAAGGTGGATGAGTACACGGGAGAATGGATTGATCCACATTTCATCGGAGGAAGATGCAAAGGACAAATTTGTGAAACAGTCCATAATTCCACAAAATGGTTTACATCCTCCGATGGAGAAAGTGTCTGCAGTCAATTGTTCACCTTGGTTGGAGGAACTTTTTTCTCTGACTCAGAGGAGATTACTTCAATGGGGTTGCCAGAAACAGGAATCAGAAGTAATTACTTTCCCTATGTATCCACAGAGGGGATTTGCAAAATGCCATTTTGCAGAAAACCGGGGTATAAGCTTAAAAATGACCTCTGGTTTCAGATTACGGACCCAGATCTGGATAAAACAGTTAGAGATCTCCCTCATATTAAGGACTGTGATCTCTCTTCATCCATTGTCACACCGGGAGAACATGCTACAGATATATCTCTGATATCGGATGTCGAAAGGATCTTAGATTATGCTCTTTGTCAGAACACATGGAGTAAAATTGAGGCTGGAGAACCAATTACCCCAGTGGATCTCAGTTATCTTGGACCAAAAAACCCAGGAGCAGGGCCGGTCTTTACCATCATTAATGGTTCACTGCATTACTTTATGTCAAAGTATCTGCGAGTAGAATTAGAAAGTCCTGTCATACCCAGAATGGAAGGCAAAGTTGCAGGAACCAGAATTGTACGGCAATTGTGGGATCAATGGTTCCCTTTCGGAGAAGTCGAGATTGGACCCAATGGTGTGTTGAAAACCAAGCAAGGGTATAAGTTCCCACTACACATCATTGGAACTGGAGAAGTGGACAATGACATCAAAATGGAAAGGATTGTCAAACACTGGGAACACCCCCATATTGAGGCTGCTCAGACATTTTTAAAAAAAGATGACACAGAAGAAGTCCTTTATTATGGCGACACCGGAGTGTCGAAAAACCCAGTTGAATTAGTTGAAGGATGGTTTAGTGGATGGAGGAGTTCCATCATGGGAGTGCTGGCTGTGATTATAGGATTTGTGATTTTAATATTTTTAATTAGGCTGATTGGGGTCTTATCCAGCCTTTTCAGACAAAAACGCAGGCCAATCTACAAATCAGACGTGGAAATGGCTCATTTCCGTTAA

SEQ ID NO: 12 - protein, 52 aa, HIV-1
LQSRPEPTAPPEESFRSGVETTTPPQKQEPIDKELYPLTSLRSLFGNDPSSQ

SEQ ID NO: 13 - protein, 511 aa, Vesicular Stomatitis Virus
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 14 - protein, 524 aa, Rabies virus
MVPQVLLFVLLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASNGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDDHVLIPEMQSSLLQQHMELLESSVIPLMHPLADPSTVFKEGDEAEDFVEVHLPDVYKQISGVDLGLPNWGKYVLMTAGAMIGLVLIFSLMTWCRRANRPESKQRSFGGTGGNVSVTSQSGKVIPSWESYKSGGEIRL

SEQ ID NO: 15 - protein, 582 aa, Nipha virus
MKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQCTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGSYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSGSESYPYDVPDYA

SEQ ID NO: 16 - protein, 521 aa, Nipha virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKK

SEQ ID NO: 17 - protein, 512 aa, Cocal virus
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 18 - protein, 512 aa, Maraba virus
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 19 - protein, 676 aa, Ebola virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSPQPPTTKPGPDNSTHNTPVYKLDISEATQVEQHHRRTDNDSTASDTPPATTAAGPLKAENTNTSKGTDLLDPATTTSPQNHSETAGNNNTHHQDTGEESASSGKLGLITNTIAGVAGLITGGRRARREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 20 - protein, 988 aa, Chikungunya virus
MSLALPVLCLLANTTFPCSQPPCTPCCYEKEPESTLRMLEDNVMRPGYYQLLKASLTCSPHRQRRSTKDNFNVYKATRPYLAHCPDCGEGHSCHSPIALERIRNEATDGTLKIQVSLQIGIKTDDSHDWTKLRYMDSHTPADAERAGLLVRTSAPCTITGTMGHFILARCPKGETLTVGFTDSRKISHTCTHPFHHEPPVIGRERFHSRPQHGKELPCSTYVQSTAATAEEIEVHMPPDTPDRTLMTQQSGNVKITVNGQTVRYKCNCGGSNEGLTTTDKVINNCKIDQCHAAVTNHKNWQYNSPLVPRNAELGDRKGKIHIPFPLANVTCRVPKARNPTVTYGKNQVTMLLYPDHPTLLSYRNMGQEPNYHEEWVTHKKEVTLTVPTEGLEVTWGNNEPYKYWPQMSTNGTAHGHPHEIILYYYELYPTMTVVIVSVASFVLLSMVGTAVGMCVCARRRCITPYELTPGATVPFLLSLLCCVRTTKAATYYEAAAYLWNEQQPLFWLQALIPLAALIVLCNCLKLLPCCCKTLAFLAVMSIGAHTVSAYEHVTVIPNTVGVPYKTLVNRPGYSPMVLEMELQSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFVVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSVTDMSCEVPACTHSSDFGGVAIIKYTASKKGKCAVHSMTNAVTIREADVEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 21 - protein, 1353 aa, MERS-CoV virus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 22 - protein, 538 aa, Homo Sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 23 - protein, 538 aa, Homo Sapiens
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRLQAIKLQTNLSAGRHPRNIQESPF

SEQ ID NO: 24 - protein, 687 aa, Homo Sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSIHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTRVGIYLCIKLKHTKKRQIYTDIEMNRLGK

SEQ ID NO: 25 - protein, 215 aa, synthetic construct
MVSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLFIQSALSKDPNEK

SEQ ID NO: 26 - protein, 124 aa, synthetic construct
GGGSSEVQLQASGGGFVQPGGSLRLSCAASGSTSRQYDMGWFRQAPGKEREFVSAISSNQDQPPYYADSVKGRFTISRDNSKNTVYLQMNSLRAEDTATYYCAFKQHHANGAYWGQGTQVTVSS

SEQ ID NO: 27 - protein, 124 aa, synthetic construct
GGGSSEVQLQASGGGFVQPGGSLRLSCAASGTTYGQTNMGWFRQAPGKEREFVSAISGLQGRDLYYADSVKGRFTISRDNSKNTVYLQMNSLRAEDTATYYCAFHDFLRMWEYWGQGTQVTVSS

(19) United States
(12) Patent Application Publication
(10) Pub. No.: US 2025/0177555 A1
(43) Pub. Date: Jun. 5, 2025

(54) TISSUE AND CELL-TYPE SPECIFIC DELIVERY OF THERAPEUTIC MOLECULES INCORPORATING VIRAL AND HUMAN FUSIOGENIC PROTEINS

(71) Applicant: THE REGENTS OF THE UNIVERSITY OF COLORADO A BODY CORPORATE, Denver, CO (US)

(72) Inventors: Xuedong LIU, Niwot, CO (US); Xiaojuan ZHANG, Boulder, CO (US); Zeyu LIU, Denver, CO (US); Quanbin XU, Denver, CO (US); Brandon BLACK, Boulder, CO (US)

(21) Appl. No.: 18/844,680

(22) PCT Filed: Mar. 9, 2023
(86) PCT No.: PCT/US2023/064057

§ 371 Date: Sep. 6, 2024

Related U.S. Application Data
(60) Provisional application No. 63/318,304, filed on Mar. 9, 2022.

(51) Int. Cl. A61K 47/69; A61K 9/50; A61K 47/64

(52) U.S. Cl. CPC A61K 47/6901; A61K 9/5068; A61K 9/5089; A61K 47/64

(57) ABSTRACT

The present invention is related to the generation and use of secreted fusogenic vesicles incorporating engineered viral glycoprotein, or engineered endogenous human fusiogenic proteins derived from human endogenous retroviruses proteins, such vesicles being generally referred to as “gectosomes.” The present invention is further related to the use of engineered gectosomes to deliver therapeutic molecules or antigens to cell and tissues in a targeted manner, as well as systems and methods for using gectosomes to screen for therapeutically relevant compounds or therapeutic antibodies.

### CROSS-REFERENCE TO RELATED APPLICATIONS

This International PCT application claims the benefit of and priority to U.S. Provisional Application No. 63/318,304, filed Mar. 9, 2022. The entire specification, claims, and figures of the above-referenced application is hereby incorporated, in its entirety by reference.

### STATEMENT OF GOVERNMENT INTEREST

This invention was made with government support under grant number AR068254 and GM144749 awarded by the National Institute of Health. The government has certain rights in the invention.

### SEQUENCE LISTING

The instant application contains contents of the electronic sequence listing (90245-00622-Sequence-Listing.xml; Size: 41,929 bytes; and Date of Creation: Mar. 9, 2023) is herein incorporated by reference in its entirety.

### TECHNICAL FIELD

The present inventive technology relates to systems, methods and compositions for the encapsulation and delivery of therapeutic molecules to target tissues and selective cell-types through secreted fusogenic vesicles. In a preferred embodiment, the secreted fusogenic vesicles incorporate engineered viral fusiogenic proteins that are designed to recruit therapeutic payloads such as small molecules, proteins, peptides, nucleic acids or ribonucleoprotein complexes (RPNs) for delivering intracellular therapeutics for gene modulation or gene therapy. In another preferred embodiment, the secreted fusogenic vesicles decorated with engineered viral glycoprotein and tethered antigens can be used to develop vaccines against viral infection or tumors by inducing immune responses or production of therapeutic antibodies. In another embodiment, engineered gectosomes with fusogenic glycoproteins can be utilized to screen and develop viral entry inhibitors or therapeutic antibodies targeting a specific viral glycoprotein for antiviral therapeutics.

### BACKGROUND

The capability to deliver macromolecules such as proteins and nucleic acids into mammalian cells is of considerable interest to researchers in both basic science and the biotech and pharmaceutical industries. Innovative methods for gene modification and interfering with mRNA expression have become nearly indispensable tools for biomedical research. All these methods rely on the delivery of nucleic acids, proteins or RPNs to the intracellular space of target cells, which is limited by the fact that the plasma and endosomal membrane is largely impermeable to biologics. For therapeutic applications, it is often desirable to deliver intercellular therapeutics to relevant cell types or tissues. However, many of existing methods such as lipid nanoparticles (LNP) are limited in this aspect due to low cell type specificity or incapable of targeting specific cell types.

Extracellular vesicles are heterogeneous, nano-sized membrane vesicles that are either constitutively or inducibly released by all cell types ranging from 40-1000 nm in size (Mathieu et al. 2019). There are two major types of EVs. Exosomes are smaller vesicles of 40-150 nm in diameter and originate from endosomal compartments known as multivesicular bodies. Microvesicles are formed and released by budding from the cell's plasma membrane and are 150-1,000 nm in diameter. Since EVs are increasingly being recognized as native means of transporting bioactive molecules between cells, there is a growing interest in exploring EVs as delivery vehicles for therapeutics.

Exosomes or microvesicles are being developed as a delivery system for intracellular therapeutic proteins or nucleic acids. The delivery efficiency of native or engineered exosomes or microvesicles is generally low since they generally lack the fusogenic proteins that are needed to release their content into the cytosol after endocytosis. Overexpression of a single viral glycoprotein from enveloped viruses such as vesicular stomatitis virus G protein (VSV-G) stimulates massive production of EVs (Mangeot et al. 2011; Zhang et al. 2020). It has been suggested that this type of vesicles can be used to delivery proteins or nucleic acids. However, EVs produced by VSV-G expression randomly encapsulates cellular proteins resulting in low specific activity of therapeutic payload and enormous cargo heterogeneity, which may limit their use in therapeutic applications.

As generally described by Liu et al., in U.S. patent application Ser. No. 17/164,624, (incorporated in its entirety herein by reference), engineered VSV-G containing a payload recruiting motif such as GFP11 promotes robust production of a type of EVs called gectosomes that encapsulate a variety of desirable therapeutic payloads. Gectosomes provide a means to transfer therapeutic proteins, nucleic acids and genome modifying machinery to recipient cells. Indeed, the inventors demonstrated that gectosomes can be utilized for delivering therapeutic nucleic acids (siRNA, miRNA, antisense oligos, sgRNA, mRNA etc.) and proteins for gene modulation and therapy.

Active loading of EVs via direct tethering to VSV-G reduces non-specific encapsulation of irrelevant cellular proteins and improves the delivery efficiency of specific payload. While VSV-G gectosomes can be effective in delivering intracellular therapeutic payloads, the broad cellular tropism of VSV-G may limit its applications where cell- or tissue-specific delivery of therapeutic payloads are more desirable.

As a result, there exists a need to identify VSV-G like proteins for similar functions but with more restricted cellular tropism that may allow tissue and cell-specific targeting by gectosomes. Additionally, there is also a need to demonstrate whether viral glycoprotein can be further modified to improve cargo loading and release. Furthermore, it is also desirable to determine whether active loading via reversible tethering to viral glycoprotein can be extended to a subset of gectosomes produced by viral glycoproteins or even human endogenous proteins. Finally, development of antiviral therapeutics rely on assays using infectious replicative viral particles under more restrictive biosafety environment. Gectosomes could provide a unique approach to overcome some of these shortcomings and yield more targeted-based approach for antiviral therapeutics discovery.

### SUMMARY OF THE INVENTION

In one aspect, the present invention is related to the generation and use of secreted fusogenic vesicles incorporating engineered viral glycoproteins, or engineered endogenous human fusiogenic proteins from human endogenous retroviruses proteins with the capability of active cargo loading, such vesicles being generally referred to as Gectosomes.

In another aspect, the invention describes a method of enhancing gectosome-mediated cargo delivery to recipient cells by incorporating a short peptide motif similar to p6Gag from HIV along with an active cargo loading motif in the glycoprotein coding sequence. In another aspect, the invention describes a new viral glycoprotein, CNV-G, or a fragment or variant thereof, from neurotropic Chandipura vesiculovirus that shares many properties with VSV-G in making gectosomes. Unlike VSV-G, CNV-G gectosomes generate a more restricted cell tropism with preferential uptake in neuronal cells. CNV-G uses cell receptor(s) distinct from VSV-G to gain entrance into cells. As a result of this key insight, the neuron-specific glycoprotein CNV-G can be engineered to encapsulate therapeutic bioactive molecules to neuronal cells and brain tissues. In another aspect, the invention describes other viral glycoproteins such as those from Carajas virus (Cara-G), Cocal virus (Coca-G), Maraba virus (Mara-G), Rabies virus (RabV-G), Ebola virus (MOKV-G), Chikungunya virus (E1E2E3), Nipah virus (NiF and NiG), SARS-CoV2 (Spike S), MERS coronavirus (Spike MS) that can be engineered to make gectosomes. Unlike VSV-G, gectosomes generated by these viral glycoproteins exhibit a more restricted cell tropism. As a result of this key insight, the cell-specific gectosomes be produced to encapsulate therapeutic payloads to be used to deliver potential bioactive macromolecules or complexes in a tissue or cell-specific manner.

In another aspect, the invention describes endogenous human fusiogenic proteins, Syncytin 1 (ERVW-1), Synctin 2 (ERVFRD-1) or HERV-K env (HERV-K-G) that share many structural elements with class I retroviral glycoproteins can be engineered to encapsulate therapeutic cargos into extracellular vesicles, or gectosome, to be used to deliver potential therapeutic compounds, such as therapeutic small molecules, proteins, nucleic acids or RNPs in a tissue, or cell-specific manner while limiting potentially adverse immunogenic responses in a human subject.

In another aspect, the invention describes a new variants of viral glycoprotein, VSV-G, or a fragment or variant thereof, from Vesicular stomatitis virus or CNV-G that allows for the production of different fusogenic gectosomes. These gectosome variants may be administered in an alternative fashion to a subject in need thereof to allow for evasion of the gectosome by the human adaptive immunity and enable repetitive dosing of different types of gectosomes.

Another aspect of the inventive technology generally includes systems, methods and compositions for an improved system of inducing an immune response to viral glycoproteins and encapsulated proteins or peptides that can elicit adaptive immunity and generate antibodies against these proteins or peptides. The gectosomes antiviral or anticancer vaccine formulation comprises (a) one of the viral glycoproteins shown to stimulate production of gectosomes; (b) antigenic proteins or antigenic peptides tethered to viral glycoprotein or complexed with the viral glycoprotein via intermediate partner with or without (c) adjuvant or innate stimulant.

In another aspect, the invention includes systems and method for using gectosomes as a high-throughput screening platform for identifying viral entry inhibitors including therapeutic antibodies using a gectosome uptake assay than using live or pseudotyped viruses. In one preferred embodiment, the invention includes systems and method for using CNV-G gectosomes as a high-throughput screening platform for identifying anti-CHPV inhibitor, and preferably anti-CHPV viral entry inhibitors, neutralizing antibodies using a CNV-G gectosome uptake assay than using live or pseudotyped viruses.

### BRIEF DESCRIPTION OF THE DRAWINGS

Additional aspects of the invention may become evident based on the specification and figures presented below.

FIG. 1. Schematic diagram of a two-hybrid gectosome system as previously described in U.S. patent application Ser. No. 17/164,624 (incorporated herein by reference). VSV-G-GFP11 (red) binds to GFP1-10-tagged Cre (orange). Gectosomes released from the producer cells are fused with the target cells to release their contents.

FIG. 2A-D. Characterization of CNV-G gectosomes by NTA and EM. (A) Schematic of two component CNV-G gectosomes. (B) Compare one component gectosomes with the two component gectosomes. Flow cytometry profiles of 293T cells transfected with one component CNV-G gectosomes (CNV-G-EGFP) or two component CNV-G gectosomes (CNV-G-GFP11/BlaM-GFP1-10). (C) NTA profiles of the size and concentrations of fluorescent two component gectosomes (D) EM analysis of two hybrid gectosomes (CNV-G-GFP11/BlaM-GFP1-10) with negative staining. Size measurement by EM.

FIG. 3A-B. Two component CNV-G gectosomes can deliver BlaM-Vpr-GFP1-10 cargo to HeLa cells and interaction of CNV-G with cargo via split GFP is required for the transfer (A) Flow cytometry analysis of 1×10⁵ HeLa cells incubated with 1×10⁸ indicated type of gectosomes for 24 hr. (B) Quantitation of BlaM activity in HeLa cells incubated with indicated gectosomes. The experiment was run in triplicate.

FIG. 4A-E. Efficiency of protein transduction by gectosomes pseudotyped with VSV-G (A or C) vs. CNV-G (B or D) in 9 cell lines and indicated mouse primary cells isolated from dissociated tissues. Percentage of BlaM positive cells are determined by flow cytometry with samples loaded with CCF dyes for measuring β-lactamase activity. Error bars, standard deviations of three replicates. (E) CNV-G Gectosomes entered human Cortical GABAergic Neurons. Human Cortical GABAergic Neurons were incubated with CNV-G-GFP11/BlaM-Vpr-GFP1-10 Gectosomes for 16 hours followed by labeling with CCF2-AM and collecting the data under fluorescence microscopy and flow cytometry.

FIG. 5A-C. Efficiency of nuclear cargo delivery by gectosomes pseudotyped with VSV-G vs. CNV-G. (A) Schematic of a ColorSwitch neuroblastoma reporter cell line. (B) Delivery efficiency of Cre by VSV-G gectosomes but not CNV-G gectosomes as determined by flow cytometry. (C) Quantitation of the delivery efficiency.

FIG. 6A-E. Robust delivery of Cre to a neural reporter cell line with the three component CNV-G gectosome system. (A) Schematic of SH-SY-5Y ColorSwitch cells. (B) Diagram of the three component gectosome system featuring inducible cargo loading with small molecule A/C dimerizer. (C) Representative images of CNV-G gectosome induced color switch. (D) Quantitation of flow cytometry analysis of the efficiency of SH-SY-5Y color switch upon incubation with indicated gectosomes. Error bars: standard deviations.

FIG. 7A-F. Flow cytometry analysis of CNS cells (˜10⁵) from nT/nG adult mouse brain incubated with (B) mock, (C) CNV-G/BlaM gectosomes (˜10⁹) or (D) CNV-G/Cre gectosomes (˜10⁹). (E) Three component CNV-G gectosomes can deliver Cre-GFP1-10 efficiently to the nucleus as determined by the efficiency of SH-SY-5Y color switch upon incubation with indicated gectosomes. Cells were isolated using a Miltenyi kit (Cat No. 130-107-677). CNV-G-GFP11/DmrC-GFP1-10/DmrA-Cre Gectosomes deliver Cre cargo into ROSAnT-nG mouse brain cells in vitro. The mouse brain cells were isolated from ROSAnT-nG mouse and incubated with CNV-G-GFP11/DmrC-GFP1-10/DmrA-Cre Gectosomes (total particle number, ˜4×10E⁹; GFP positive particle number, ˜4×10E⁸; CNV-G-GFP11/BlaM-Vpr-GFP1-10 as control) for 48 hours in 12-well plates (˜1×10E⁵ cells each well) followed by flow cytometric analysis. The statistic data showed the percentage of the color conversion (Data are the mean±SD (n=3)). (E) and (F) CNV-G-GFP11/DmrC-GFP1-10/DmrA-Cre Gectosomes deliver Cre cargo into ROSAnT-nG mouse tissues. (E) CNV-G-GFP11/DmrC-GFP1-10/DmrA-Cre Gectosomes deliver Cre cargo into ROSAnT-nG mouse brain through intracranial administration. CNV-G-GFP11/DmrC-GFP1-10/DmrA-Cre Gectosomes were generated and then purified through ultrafiltration. The Gectosomes (˜1×10E⁹/mouse) were intracranially injected into mice (n=3). After six weeks post injection, the mice were sacrificed to take out the brain followed by applying the tissues to Frozen Section Technique and Microscopy to observe the color switch in tissues. (F) data quantitation.

FIG. 8A-D. Delivering CNV-G gectosomes encapsulated with catalase can protect cells from superoxide induced apoptosis. (A) 293T cells transfected with CNV-G and Catalase to produce fluorescent gectosomes. (B) NTA analysis of fluorescent CNV-G gectosomes in the supernatant. (C) Neuro-2A cells incubated with CNV-G gectosomes with catalase before treated with 500 uM of H₂O₂ for 24 hr. Effects of apoptosis was measured by flow cytometry analysis. (D) Quantitation of the effects of gectosome delivery of catalase on apoptotic responses as measured by Annexin V/PI staining.

FIG. 9A-F. Apoptotic effect of U87 GBM cells treated with AVIL interference RNAs delivered by CNV-G gectosomes, exosomes or lipids. 6×10⁵ cells were incubated with (A) Control supernatant; (B) CNV-G/BlaM (˜1010) gectosomes. (C) CNV-G/Cre/siAVIL (˜1010). (D) CNV-G/LwaCas13a/siAVIL (˜1010). (E) Exosomes/siAVIL (˜1010) (F) RNAiMax siAVIL for 90 hr and harvested for flow cytometry analysis after staining with Annexin. 0.5 nmol of siAVIL was used or loaded. The particle numbers were measured by Nanosight.

FIG. 10A-B. (A) Schematic diagram of a two-hybrid gectosome system for mRNA loading and intercellular mRNA transfer. (B) RT-PCR analysis of gectosomes purified from 293T cells transfected with indicated combination of expression vectors. MCP: MS2-coat protein; MS2: MS2 coat protein binding sequence. CFP: cyan fluorescence protein. M:1 kb DNA ladder.

FIG. 11. Displaying CD47 on the surface of CNV-G gectosomes by co-encapsulation slows down depletion of gectosomes upon incubation with mouse RAW264.7 macrophage cells.*** p<0.01, student t-test

FIG. 12A-E. Pharmacokinetics and biodistribution analysis of levels of VSV-G and CNV-G gectosomes in blood and tissues. (A) Schematic diagram of experiment process of pharmacokinetic assay and biodistribution analysis of CNV-G/DmrC/DmrA-nanoLuc and VSV-G/DmrC/DmrA-nanoLuc Gectosomes in mouse. (B) NanoLuc activity of CNV-G/DmrC/DmrA-nanoLuc and VSV-G/DmrC/DmrA-nanoLuc Gectosomes applied to mice before injection. (C) Pharmacokinetic assay result shows the nanoLuc activity per microliter serum post injection change over time. (E) and (F) Relative nanoLuc activity normalized to total tissues at 1 h or 24 h post injection of CNV-G-GFP11/DmrC-GFP1-10/DmrA-nanoLuc or VSV-G-GFP11/DmrC-GFP1-10/DmrA-nanoLuc Gectosomes into mice (n=4).

FIG. 13A-B. CNV-G and VSV-G uses different receptors to gain entrance into the cell. LDLR knockdown significantly decreased the percentage of switched cells induced by VSV-G Gectosomes but not CNV-G Gectosomes. 293ColorSwitch cells treated with siLDLR for 48 hours were incubated with CNV-G-GFP11/DmrC-GFP1-10/DmrA-Cre or VSV-G-p6-Gag-GFP11/DmrC-GFP1-10/DmrA-Cre Gectosomes for 48 h and then submitted to flow cytometric assay to detect the percentage of switched cells. C shows RT-qPCR result of LDLR knockdown efficiency.

FIG. 14A-B. Cara-G pseudotyped gectosomes can transduce BlaM-GFP1-10 to various cancer cell lines. Noted that PANC-1 and HaCat cell lines can be transferred with Cara-G but not with VSV-G gectosomes.

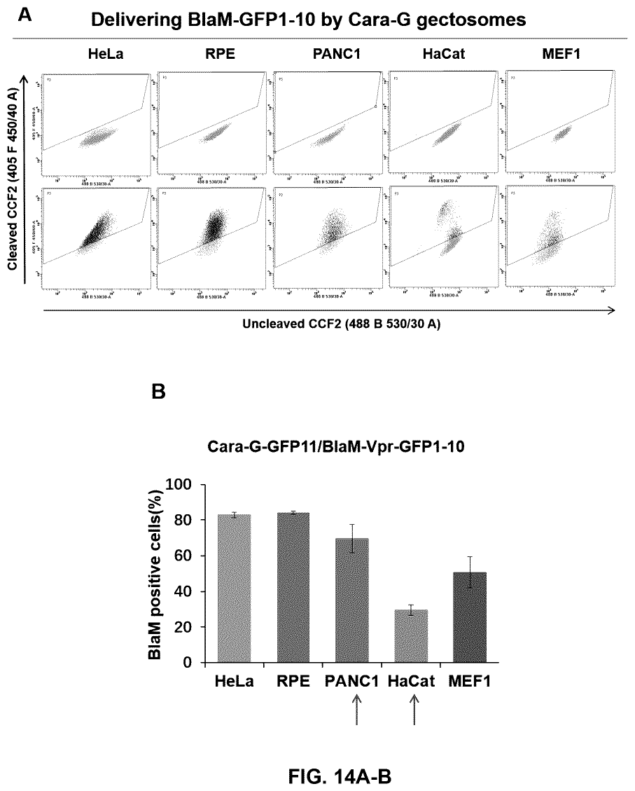

FIG. 15A-B. (A) Uptake of VSV-G vs. VSV-G-NJ gectosomes carrying Cre-GFP1-10 in 293ColorSwitch cells in the presence or absence of a VSV-G neutralizing antibody (8G5F11). 10⁸ vesicles were applied to 10⁵ cells along with control IgG or 8G5F11 antibody and analyzed after 48 hr. Neutralizing antibody 8G5F11 prevents VSV-G gectosome uptake but not VSV-G-NJ gectosome uptake. (B) Uptake of VSV-G vs. CNV-G gectosomes carrying Cre-DmrA/DmrC-GFP1-10 in Mouse neuron N2A-ColorSwitch cells in the presence or absence of a CNV-G neutralizing antiserum. 10⁸ vesicles were applied to 10⁵ cells along with control IgG or CNV-G antiserum (1.300) and analyzed after 48 hr. Neutralizing antiserum against CNV-G prevents CNV-G gectosome uptake but not VSV-G gectosome uptake.

FIG. 16. Pilot HTS of 320 chemical compounds for inhibitors of CNV-G gectosome cell entry. Solid blue line represents mean percentage of cells that are positive for GFP as a result of Cre delivery. Dash lines are mean±3SDM(σ). Chloroquine (blue circles) was used and positive controls and vehicle as negative controls (red diamonds). Each dot represents a compound with a randomly assigned color.

FIG. 17A-C. Dose-response analysis of CNV-G gectosome inhibition by Latrunculin B (IC50=1.96 μM) (A) or U-73122 (IC50=3.03 μM) (B). (C) Latrunculin B blocks CNV-G gectosome-mediated Cre transfer but has no effect on VSV-G gectosome-mediated Cre transfer while chloroquine can inhibit both. Data are presented as mean±STDEV. Statistical significance was assessed by two-way ANOVA test. N.S. not significant. p<0.001.

FIG. 18. The selection and determination strategies. The first selection is the determination of the percentage of color switched cells. And then cell viability is detected to rule out the compounds inducing cell death or apoptosis. Nine compounds from the first and second round selection are submitted to dose response selection.

FIG. 19A-C. (A) and (B) Dose response curve of Milterfosine and Berbamine HCl. The series dilution of Milterfosine and Berbamine HCl were incubated with the mixture of HeLa-ColorSwitch cells and CNV-G-GFP11/DmrC-GFP1-10/DmrA-Cre Gectosomes and then the treated cells were submitted to flow cytometry analysis to figure out the percentage of the switched cells. IC50 of Milterfosine is 5.1 μM and IC50 of Berbamine HCl is 2.4 μM. Data are the mean±SD (n=3). (C) The effect of Milterfosine and Berbamine HCl on Cre cargo delivery from CNV-G-GFP11/DmrC-GFP1-10/DmrA-Cre or VSV-G-p6Gag-GFP11/DmrC-GFP1-10/DmrA-Cre Gectosomes in 293ColorSwitch cells. Data are the mean±SD (n=3).

FIG. 20A-C. Induction of host adaptive immune responses by gectosome delivery of antigens. Mice were administrated with 2×10¹⁰ CNV-G-GFP11/DmrC-GFP1-10/NanoLuc-DmrA via intravenous or intramuscular injection every three weeks. Blood was collected every two weeks and tested for presence of antibodies against CNV-G gectosomes (A), recombinant GFP (B) or NanoLuc (C).

FIG. 21A-B. Presence of CNV-G on the surface of CNV-G gectosomes. 1×10⁹ CNV-G gectosomes were stained with an anti-CNV-G serum (1:300) or a control serum (1:300) overnight at 4 degree. An Alexa Fluor®488-Anti-Mouse secondary antibody (1:1000) was added to each sample and incubated at room temperature for 3 hours. The stained samples were analyzed by Laser Flow Analyzer (NanoFCM). Flow cytometry profiles of CNV-G gectosomes stained with (A) an anti-serum against CNV-G and (B) against a control antiserum.

FIG. 22A-B. Low immunogenicity dimerization modules for encapsulating cargos of gectosomes and a new chemical inducible cargo loading method. (A) Relative NanoLuc activities in gectosomes collected from 293T cells transfected with indicated pair or single expression vectors 48 hr after transfection. (B) Relative Nanoluc activities in gectosomes collected from 293T cells transfected with VSV-G-CA14 plus Cre-Nluc-DB21 or Cre-Nluc-DB21 alone treated with or without CBD (10 μM) at 24 hr or 48 hr after transfection..

### DETAILED DESCRIPTION OF THE INVENTION

Generally, the inventive technology includes systems, methods and compositions for making a specific type of EVs called Gectosomes, which facilitate cargo loading and their endosomal escape simultaneously. In one embodiment, Gectosomes contain two major components: an engineered viral fusiogenic protein, such as VSV-G variants, CNV-G, SARS-G or S, CARA-G RAB-G, NIV-G, COCA-G, MARA-G, MOKV-G, CHIKV-G, MERS-G or M, as described herein, and the cargo of interest tethered to one another via a split complement system, such as a split protein system and/or a protein-protein motifs. In one embodiment, the split complement system, such as a split protein system and/or a protein-protein motifs includes a Split-Fluorescent Proteins (SFPs) including Split-Green Fluorescent Proteins (GFP). Complementation of split-GFP enables more efficient loading of the specific cargo and purification of desired fluorescent Gectosomes. As detailed below, such engineered Gectosomes may be configured to deliver proteins or protein/RNA complexes designed to modify genotypes in mammalian cells in vitro, ex vivo and in vivo.

The inventive technology further includes systems, methods and compositions for making a specific type of EVs called Gectosomes, which facilitate cargo loading and their endosomal escape simultaneously. In one embodiment, Gectosomes contain two major components: an engineered human fusiogenic protein, such as SYN1-G, SYN2-G, and HERV-K-G, as described herein, and the cargo of interest tethered to one another via a split complement system, such as a SFPs including GFP. Complementation of split-GFP enables more efficient loading of the specific cargo and purification of desired fluorescent Gectosomes. As detailed below, such engineered Gectosomes may be configured to deliver proteins or protein/RNA complexes designed to modify genotypes in mammalian cells in vitro, ex vivo and in vivo. The application of endogenous human-derived G-proteins to produce gectosomes may reduce or eliminate adverse immunogenic responses in a subject.

Additional embodiments of the invention may also include a programmable or engineered gectosome vesicle incorporating viral and/or human fusiogenic proteins that is configured to selectively encapsulate and deliver specific nucleic acids, and/or other small molecules such as peptides or complexes of the same, generally referred to as target molecules, to a target recipient cell in a predetermined manner through the use of a split complement system, such as a split protein system and/or a protein-protein motifs. For example, in one preferred embodiment, a split protein system selected from the group consisting of: a split GFP system, a spit CFS system, a NanoBiT (Promega) split ubiquitin system, a split beta-gal system, a split luciferase system, a split mCherry system and the like.

In another embodiment, the invention may include a three-components gectosome-mediated delivery system, which may further be tissues=and/or cell-specific. In this embodiment, a first fusion peptide may be provided having a membrane-fusion moiety, such as a CNV-G, Cara-G, VSV-G variants, RAB-G, NIV-G, COCA-G, MARA-G, MOKV-G, CHIKV-G, S, SM, SYN1-G, SYN2-G, and HERVK-G, or fragment thereof, a first component of a split complement system, such as a split-GFP system. A second fusion peptide may be provided having a second component of the split complement system, and a first dimerization domain. A third fusion peptide having a second dimerization domain and a target molecule may further be provided. In this embodiment, the membrane-fusion moiety is configured to be anchored to a cell membrane that forms an extracellular vesicle (EV) the first, second, and third fusion peptides form a trimeric complex thereby loading said target molecule, which may be an oligonucleotide, peptide, or peptide nucleotide complex among others, into said EV forming a gectosome for delivery to a target cell. This gectosome may further optionally display a CD47 peptide to suppress clearance by the gectosome by macrophages.

Dimerization domains of interest include, but are not limited to, protein domains of the iDimerize inducible homodimer (e.g., DmrB) and heterodimer systems (e.g., DmrA and DmrC) and the iDimerize reverse dimerization system (e.g., DmrD) (see e.g., Clontech.com Cat. Nos. 635068, 635058, 635059, 635060, 635069, 635088, 635090 and 635055) (See (Clackson et al. 1998); (Crabtree and Schreiber 1996); (Jin et al. 2000); (Muthuswamy, Gilman, and Brugge 1999)), or nanobody based chemical dimerization module (Kang et al. 2019).

Another embodiment of the current inventive technology generally includes systems, methods and compositions for an improved system for the encapsulation and targeted delivery of therapeutic molecules to neuronal cells, such as brain-derived neurotrophic factor (BDNF), through gectosome of the invention, such as for example a secreted fusiogenic viral glycoprotein CNV-G containing gectosomes, among others. In one preferred embodiment, one or more gectosomes of the invention may be programed to effectuate the high-efficient intercellular transfer of their cargo to neuronal tissues or cell lines in vivo and in vitro, as well as select somatic tissue in live animals. In certain embodiments, the invention allows for the high-level purified of homogenous microvesicles with respective to their target cargo, thereby reducing undesirable bioactive contaminants.

Another embodiment of the invention includes generalizable methods for active loading and purification of highly specific gectosomes, which are capable of effectively delivering genome-modifying tools to target cells in vitro, ex vivo and in vivo. In one preferred embodiment, such gectosomes and are designed to co-encapsulate CNV-G, Cara-G, VSV-G variants, RAB-G, NIV-G, COCA-G, MARA-G, MOKV-G, CHIKV-G, S, MS, SYN1-G, SYN2-G, and HERVK-G with bioactive proteins, mRNAs, and protein-RNA complexes, that can execute designed modifications of endogenous genes in cell lines in vitro, ex vivo and somatic tissues in vivo, allowing for the targeted gectosome-mediated delivery of therapeutics for a wide range of human diseases.

In another embodiment, the invention may include systems, methods and compositions for making programmable, highly fusogenic gectosomes, which may be uses as vehicles for the dose-controlled delivery of specific therapeutic or diagnostic agents, in vitro and in vivo, further incorporating one or more targeting motifs that may enhance cargo delivery to a target tissues or cell. In one preferred embodiment, a CNV-G protein, or fragment thereof, may be coupled with a protein sequence that increases delivery efficiency of the desired interacting partners into a CNV-G containing vesicles. In one preferred embodiment, this increase cargo delivery efficiency may be accomplished through inserting a peptide, or peptide fragment containing a Gag-motif, such as a p6^{Gag} peptide domain from HIV-1 into CNV-G protein. Viral glycoproteins tagged with the p6^{Gag} motif may promote vesicle production in producer cells and cargo escape from the endosome once a gectosomes enters a target cell.

In another embodiment, the invention may include systems, methods and compositions for making programmable, highly fusogenic gectosomes, which may be used as vehicles for the dose-controlled delivery of specific agents, such as therapeutic or diagnostic agents, in vitro and in vivo, further incorporating one or more target proteins that may enhance cargo delivery to a target tissues or cell. In one preferred embodiment, a human-derived fusiogenic peptide, such as a SYN1-G, SYN2-G, and HERVK-G protein, or fragment thereof, may be coupled with a protein sequence that increases delivery efficiency of the desired interacting partners into a SYN1-G, SYN2-G, and HERVK-env containing vesicles. In one preferred embodiment, this increased cargo delivery efficiency may be accomplished through the expression of a peptide, or peptide fragment containing a Gag-motif, such as an p6^{Gag} peptide domain from HIV-1, with a SYN1-G, SYN2-G, and HERVK-G protein. Linking of the p6^{Gag} peptide with a SYN1-G, SYN2-G, and HERVK-G protein may promote cargo escape from the endosome once a gectosomes enters a target cell.

In another embodiment of the invention, a split component system, such as split GFP system may be used as a driver between a fusiogenic peptides, such as CNV-G, Cara-G, VSV-G variants, RAB-G, NIV-G, COCA-G, MARA-G, MOKV-G, CHIKV-G, S, SM, SYN1-G, SYN2-G, and HERVK-G, and the desired cargo proteins as such fluorescent gectosomes may be efficiently formed during shedding to the extracellular space. In another embodiment, gectosomes with a desired cargo may be purified by fluorescence-activated cell sorting (FACS) to obtain nearly homogeneous particle populations. In additional embodiments, the invention may allow for RNA interference, gene editing, and RNA ablation with designed CNV-G, Cara-G, VSV-G variants, RAB-G, NIV-G, COCA-G, MARA-G, MOKV-G, CHIKV-G, S, SM, SYN1-G, SYN2-G, and HERVK-G gectosomes, for example in targeted neuronal cells. Additional embodiment s of the invention may include the clinical application of CNV-G, Cara-G, VSV-G variants, RAB-G, NIV-G, COCA-G, MARA-G, MOKV-G, CHIKV-G, S, SM, SYN1-G, SYN2-G, and HERVK-G gectosomes for therapeutics by achieving transient delivery of therapeutic compositions, such as, CRISPR editing machinery, antisense oligos, siRNA, translatable mRNAs, to target neuronal cells.

In one preferred embodiment, the inventive technology includes systems, methods, and compositions for the in vitro and/or in vivo generation of engineered or programmable fusogenic secreted vesicles that may be configured to be loaded with one or more specific target molecules. As generally shown in the Figures, in one embodiment, a donor cell may be engineered to generate fusogenic secreted vesicles having a targeting moiety expressed on the surface of the vesicles. This targeting moiety may include a protein, or protein fragment that may bind to a moiety present on a target or recipient cell. As described below, in one preferred embodiment an engineered fusogenic secreted vesicles may include a fusiogenic protein, such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein, that has been engineered to further include an interacting moiety that can be recognized by encapsulated proteins directly or indirectly, through a dimerization system, to form interacting complexes. This interacting moiety may further be configured to be recognized by encapsulated proteins that may further bind proteins, protein fragments, nucleic acids, and/or small molecules as generally described herein.

In one embodiment the invention may further include methods for the generation and/or loading of engineered fusogenic secreted vesicles, such as gectosomes, with one or more proteins, nucleic acids, and/or small molecules as generally described herein. In one preferred embodiment, an engineered fusogenic secreted vesicle may be loaded with a target cargo through electroporation, liposomal transfection or fusion with other types of vesicles among other mechanisms known in the art. The invention may further include systems, methods and compositions for the generation of engineered fusogenic secreted vesicles, generally referred to as gectosomes, having a fusiogenic protein, such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein and/or other microvesicle producing proteins that contain an interacting moiety that can be recognized by encapsulated proteins that bind proteins/peptides, nucleic acids or small molecules as generally outlined herein.

In a preferred embodiment the invention may include systems, methods and compositions for making programmable, highly fusogenic gectosomes, which may be uses as vehicles for the dose-controlled delivery of specific pharmacological agents in vitro and in vivo, further incorporating one or more target proteins that may enhance cargo delivery to a target cell. In one preferred aspect, a fusiogenic protein, such as a fusiogenic protein, such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein, or a fragment thereof may be coupled with a protein sequence element that increases delivery efficiency of the desired interacting partners into gectosome. In one preferred aspect, a this increase cargo delivery efficiency may be accomplished through the expression of a peptide, or peptide fragment containing a p6^{Gag} peptide domain with a fusiogenic protein. Co-expression of the p6^{Gag} peptide with a fusiogenic protein, such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein, may promote cargo escape from the endosome once a gectosomes enters a target cell. In one embodiment, expression of fusiogenic protein, such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein, and a p6^{Gag} peptide may be from the same expression cassette forming a fusion protein. In this embodiment, the fusiogenic protein, such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein, and a p6^{Gag} peptide may be coupled with a linker or other spacer element, or a tag, such as a myc-tag. The p6^{Gag} peptide may include a domain directed to the Endosomal Sorting Complex Required for Transport (ESCRT), with binding sited for ESCRT-1, ALIX and Vpr.

The invention may further include systems, methods and compositions for the generation of engineered fusogenic secreted vesicles having human gag-like endogenous proteins and an interacting moiety that can be recognized by encapsulated proteins that bind proteins/peptides, nucleic acids or small molecules as generally outlined herein. In a preferred embodiment, an engineered fusogenic secreted vesicle may include human gag-like endogenous proteins and an interacting moiety that can perform perturbation of gene functions such as Cas9, dCas9, SaCas9, dSaCas9, LwaCas13, RfxCas13d, Cas13, C2c1, C2C3, C2c2, Cfp1, MAD7, CasX, base editor, CRISPRi, CRISPRa, CRISPRX, CRISPR-STOP and base editors as generally described herein.

As noted above, in one preferred embodiment an engineered fusogenic secreted vesicles may include a fusiogenic protein, such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein, that has been engineered to further include an interacting moiety that can be recognized by encapsulated proteins directly or indirectly to form interacting complexes. In one preferred embodiment, one or more target molecules may be selected through direct and/or indirect interaction with a fusogenic proteins, such as viral glycoproteins. For example, in certain preferred embodiments, VSV-G variants or like proteins in Ebola, Rabbies, Heptatis C, Lymphocytic choriomeningitis (LCMV), Autographa californica nuclear polyhedrosis virus (AcMNPV) and Chandpura (CNV) may be utilized to produce programmable ectosomes that can be used for transferring proteins, RNAi and/or genome editing agents as generally described herein. As described herein, such fusogenic proteins may not only promote production of programmable ectosomes but may also exhibit a distinct host and/or cell range. For example, in one embodiment, a viral G protein, such as CNV-G, Cara-G, VSV-G variants, RAB-G, NIV-G, COCA-G, MARA-G, MOKV-G, CHIKV-G, S, SM, SYN1-G, SYN2-G, and HERVK-G may be used to generate programmable ectosomes. As such, the inventive technology allows for the generation of cell, tissue, and/or organisms' specific programmable secreted fusogenic ectosome vesicles.

In another embodiment, the inventive technology may further include the generation of secreted fusogenic vesicles, such as gectosomes, that may further be introduced to proteins, nucleic acids or small molecules of the type generally described herein. In one embodiment, secreted fusogenic vesicles that may be electroporated or transfected with proteins, nucleic acids or small molecules as generally identified herein. In one embodiment, self-complementing split fluorescent proteins (SFPs) may be used to generate two-component fluorescent gectosomes with recombinant fusiogenic variants.

SFPs are a protein complex composed of two or more protein fragments that individually are not fluorescent, but, when formed into a complex, result in a functional (that is, fluorescing) fluorescent molecule. Complementary sets of such fragments are also known as a SFP system, and typically include a SFP detector (comprising 9-10 strands of an 11 β-barrel fluorescent protein) and one or two SFP tags (comprising the remaining strands of the fluorescent protein). The SFP detector complements with the heterologous SFP tag (or tags) to form a functional (that is, fluorescing) fluorescent protein. Thus, an SFP tag and the complementary SFP detector are two complementing fragments of an SFP. In certain embodiment, a split GFP system may include a detector of GFP1-10 and a GFP11 tags. Polypeptides comprising Split-GFP fragments are known to the skilled artisan and further described herein. See, e.g., U.S. Pat. App. Pub. No. 2005/0221343 and Int. Pat. App. Pub. No. WO/2005/074436, and (Cabantous, Terwilliger, and Waldo 2005),; (Cabantous and Waldo 2006). Other variations are also available; see, e.g., U.S. Pat. App. Pub. No. 2005/0221343. The polypeptides comprising complementing Split-GFP fragments disclosed herein will form a functional GFP molecule when complemented.

Construction of a test protein fused to a SFP tag or SFP detector is typically accomplished via cloning of the nucleic acid encoding the test protein into a nucleic acid construct encoding the SFP tag or SFP detector. SFPs, SFP systems, a number of specifically engineered tag and detector fragments of a SFP, such as split GFP systems, as well as DNA constructs and vectors use thereof are disclosed herein and known to the skilled artisan. See, e.g., U.S. Pat. App. Pub. No. 2005/0221343; Int. Pat. App. Pub. No. WO/2005/074436; (Cabantous, Terwilliger, and Waldo 2005); (Cabantous and Waldo 2006)) Typically, the SFPs include two SFP fragments, such as a SFP tag (typically corresponding to GFP11) and a SFP detector (typically corresponding to GFP1-10). Other SFPs are disclosed herein.

In certain embodiments, fusiogenic peptides, such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein, may be generated. Such peptides may contain a short peptide tag derived from a split protein system which enables them to form stable complex with any protein(s) that is fused to its complementary fragment. For example, in one embodiment a VSV-G was fused to a 16 amino acid peptide tag (GFP11). This fusion generates fluorescence when co-expressed with its complementary fragment, GFP1-10. In one preferred embodiment, an amino acid peptide tag GFP1-10 may be fused with a target molecule, such as a protein that can modify gene expression or have some other phenotypic or therapeutic effect on the target cell. In this exemplary embodiment, the GFP1-10-fusion may be co-expressed with, for example, CNV-G-GFP11, resulting in the transfer functionality from donor cells to recipient cells with high fidelity. In another exemplary embodiment, the GFP1-10-fusion may be co-expressed with, for example, SYN1-G-, SYN2-G-, or HERVK-G-GFP11, resulting in the transfer functionality from human donor cells to recipient cells with high fidelity.

As noted above, transducing nucleic acids or proteins into live cells to alter cellular function is crucial for studying gene function in the research space and growing increasingly more consequential to therapeutics due to the explosion of biologics as a compelling therapeutic modality. Viral-mediated gene transfer for overexpression, RNA interference, and gene editing works well for research but poses safety concerns for therapeutics. For decades, liposomes have been the preferred delivery vesicle for drugs and other cargo of interest. Despite their intense research development, major barriers including low stability, short circulation life, endosome degradation, high toxicity in vivo, inefficient loading for hydrophobic drugs, and difficulty in targeting remain to be overcome. EVs are heterogeneous nano-sized membrane vesicles constantly released by all cell-types. EVs have been classified either as exosomes or microvesicles, also known as ectosomes. Microvesicles are formed and released by budding from the cell's plasma membrane and are 150-1,000 nm in diameter. Exosomes are smaller vesicles of 40-150 nm in diameter and originate from endosomal compartments known as multivesicular bodies. While it has been well documented that exosomes can encapsulate small RNAs, its capability of carrying larger mRNA is still unproven. Furthermore, active loading of EVs with pre-determined cargoes and purifying them to homogeneity are required for development of EVs as therapeutics.

In one specific embodiment, AGO2 or LwaCas13, a known essential components of the RNA-induced silencing complex (RISC) that binds small interfering RNAs (siRNAs) and other noncoding RNA including microRNAs (miRNAs) and Piwi-interacting RNAs (piRNAs), may be fused with a split components system, such as GFP1-10 and co-introduced with a fusiogenic peptide-GFP11 fusion protein along with a target interfering RNA molecule, such as a short-hairpin RNA (shRNA). In this embodiment, the GFP1-10-AGO2 or a GFP-10- LwaCas13 construct may be co-introduced with fusiogenic peptide-GFP11 and a target interfering RNA (RNAi), such as a hpRNA, to a recipient cell through direct transfection, for example in an in vitro model. In alternative embodiments, the GFP1-10-AGO2 or LwaCas13 construct may be co-introduced with fusiogenic peptide -GFP 11 and a target shRNA or other interfering RNA, such as a CRISPR RNA (crRNA) through the introduction of programmable gectosomes from a donor cell to a recipient cell, in an in vitro or in vivo system as generally outlined herein. In each of the embodiments described above, the target RNAi molecule, such as a shRNA may be configured to inhibit expression of a specific endogenous gene in the target cell. Alternatively, in certain preferred embodiments, the target RNAi molecule, such as a shRNA may be configured to inhibit expression of a specific exogenous gene, such as an essential bacterial or pathogen gene or other transgene.

In another embodiment, a target mRNA molecule for a select peptide, may be delivered to a target cell through the gectosomes of the invention. In one preferred embodiment a target peptide, such as L7Ae, having an RNA binding domain/motif may be coupled with a component of a split components system, such as GFP1-10. This fusion peptide may be co-expressed with a second fusion peptide having a membrane-binding motif, such as a fusiogenic peptide that is coupled with a complementary component of the split component system of the first fusion peptide. A target RNA molecule may further be co-expressed with the first and second fusion peptides and may bind to the RNA binding domain of the target peptide domain.

In the preferred embodiment, a target mRNA may include a coding region configured to be coupled with BoxCD binding domain that may interact with the RNA binding domain of a target peptide, such as L7Ae. Again, a Cre mRNA having a BoxCD binding domain may bind to a corresponding BoxCD RNA binding domain of the target protein L7Ae. The L7Ae-GFP-1-10 may complement with a corresponding split protein of the fusiogenic peptide-GFP11 fusion peptide that is anchored to the cell membrane from which an EV can be formed as generally described herein. In this configuration, the Cre mRNA is loaded into the gectosome in a producing cell and may further be isolated and/or be introduced to a target call in vitro or in vivo, such that the mRNA is introduced into the intracellular compartment of the target cell and subsequently translated.

In another embodiment, mRNA molecules can be incorporated into gectosomes via active loading of gectosomes and detected in secreted gectosomes. For example, in one embodiment, the inventions describes a two-hybrid gectosome system for mRNA loading and intercellular mRNA transfer. In this embodiment, a fusion peptide containing a MS2-coat protein and a component of a split components system may be co-expressed with a second fusion protein having a fusiogenic peptide such as CNV-G, Cara-G, VSV-G variants, RAB-G, NIV-G, COCA-G, MARA-G, MOKV-G, CHIKV-G, S, SM, SYN1-G, SYN2-G, and HERVK-G as described herein, fused with a complementary components of the split component system, in this instance a split GFP system. The fusiogenic peptide-GFP-11 fusion protein is anchored to a cell membrane that forms an extracellular vesicle (EV). The reporter RNA molecule binds to the MS2-coat protein target peptide and the GFP-1-10 portion of the split GFP system binds to is corresponding GFP-11 components thereby loading the mRNA bound target molecule into said EV forming a gectosome for delivery of the mRNA molecule to a target cell.

The invention further includes systems, methods and compositions for an improved system for the encapsulation and delivery of target genome-editing molecules to recipient cells through secreted fusogenic ectosome vesicles, such as gectosomes. In one preferred embodiment, the invention includes a programmable fusogenic ectosome vesicle, such as a gectosome, that is configured to selectively encapsulate and deliver specific genome-editing proteins to a recipient cell in a predetermined manner. Examples, may include, but not be limited to: Meganucleases (MGN), Zinc-F̅inger N̅ucleases (ZFN), Transcription Activator-Like Effector Nucleases (TALENs) and the like.

In a preferred embodiment, the inventive technology may include the systems, methods and compositions for the generation of secreted fusogenic vesicles that contain Cas9 and/or Cas13, or other genome editing proteins related to the genome editing process known as Clustered Regularly Interspaced Short Palindromic Repeats (CRISPR). In this preferred embodiment, a programmable fusogenic ectosome vesicle, such as a gectosome, may be configured to selectively encapsulate and deliver CRISPR ribonucleoproteins (RNP) to a target cell and mediate genome editing. In one specific embodiment, Cas9/sgRNA RNP, a known essential component of CRISPR genome editing, may be fused with tag, such as split complement protein system, such as GFP1-10 and co-introduced with fusiogenic peptide-GFP11. In this embodiment, the GFP1-10-Cas9/sgRNA RNP construct may be co-introduced with fusiogenic peptide-GFP11 to a recipient cell through direct transfection, for example in an in vitro model. In alternative embodiments, the GFP1-10-Cas9/sgRNA RNP construct may be co-introduced with fusiogenic peptide-GFP11 through the introduction of programmable gectosome from a donor cell to a recipient cell, in an in vitro or in vivo system as generally outlined herein. In each of the embodiments described above, the sgRNA, or single guide RNA molecule, may be configured to target a specific endogenous gene in the target. Alternatively, in certain preferred embodiments, the target sgRNA molecule may be configured to inhibit expression of a specific exogenous gene, such as an essential bacterial or pathogen gene or other transgene.

The inventive technology may further include the generation of engineered fusogenic secreted vesicles through the action of human Gag-like proteins. In this embodiment, one or more human Gag-like endogenous proteins may be coupled with an interacting moiety, such as GFP11 or a similar tag that can be recognized by encapsulated proteins that bind proteins/peptides, nucleic acids or other small molecules as generally described herein.

The invention may further include systems and methods and compositions for the use of engineered fusogenic secreted vesicles, such as gectosomes for the treatment of a disease condition. Examples may include, but not be limited to treatment and/or prevention of cancer, autoimmune conditions, vaccines, and organ and/or cell transplant rejection. In one preferred embodiment, a therapeutically effective amount of engineered fusogenic secreted vesicles, as generally described herein, may be introduced to a recipient cell exhibiting a disease condition, such that the action of the engineered fusogenic secreted vesicles may alleviate and or prevent a disease condition. In additional embodiments, engineered fusogenic secreted vesicles, such as gectosomes, may be used to increase host immunity and/or metabolic fitness or even replace missing or defective cell pathways in a recipient cell.

In additional embodiment, the invention may include the generation of high-efficiency persistent gectosomes that may resist clearance by the immune system, for example through the expression of surface biomarkers that prevent immune system clearances. In one preferred embodiment, such a high-efficiency persistent gectosomes that may resist clearance by the macrophages through the overexpression and presentation of CD47 proteins on the surface of the gectosomes.

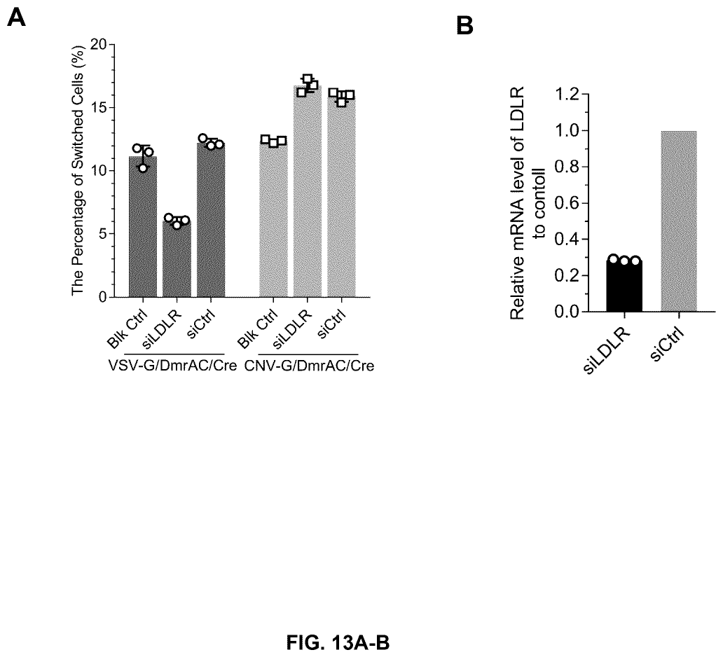

In another embodiment, the present invention may include the use of gectosomes as a High Throughput Screening (HTS) system. In one embodiment, a gectosomes, and in particular a gectosome incorporating a viral fusiogenic or glycoproteins peptide may be use in a High Throughput Screening system to screen for small-molecule or other biologic inhibitors and probes of viral replication, and in particular inhibitor of viral fusion and entry into a target host cell. For example, the critical path for identifying anti-CHPV chemical probes and molecular targets that modulate CHPV cellular entry is presented in FIG. 13. In one embodiment, a screen may identify a lead compound that can prevent CNV-G gectosomes or CNV-G pseudotyped lentiviral infection.

On one embodiment, the invention may include the use of gectosomes, and in particular CNV-G gectosome to screen and identify anti-CHPV compounds that selectively block the CNV-G function. As described herein, the present inventors have demonstrated that CNV-G gectosomes encapsulated with Cre recombinase can be efficiently transferred to the recipient cell that harbors LoxP-flanked fluorescent reporter. A validated HTS platform may incorporate CNV-G/Cre gectosomes to perform an HTS screen of possible inhibitor compounds to identify diverse chemotypes that block the CNV-G gectosome uptake and cargo release. Hits identified from a primary HTS may be confirmed first by dose-response analysis and orthogonal counter screen assays to eliminate false positives as well as chemically intractable inhibitors. Hit specificity may be evaluated further by VSV-G gectosomes and VSV-G or CNV-G pseudotyped lentiviral reporter viruses. The cytotoxicity of hits can be determined to prioritize high potency (IC50<5 μM) and low toxicity (IC50>10 μM) compounds. SAR analysis may further be performed to obtain analogs with better potency and selectivity for the mechanism of action studies.

The present inventors gectosome-based HTS may allow the identification of gectosome uptake and cargo release inhibitors. The follow-up assays can verify identified hit as authentic viral entry inhibitors and pinpoint which cellular processes controlled by, for example CNV-G are targeted by the compounds. A time-of-addition (TOA) variation assay with CNV-G pseudotyped virus can be performed to confirm the entry inhibitory profiles. Immunofluorescence, TIRF microscopy and live-cell imaging can further be used to quantify CNV-G pseudotyped viral entry processes with inhibitors. As some hits emerge from the screen of FDA-approved drug libraries, it can be determined whether these hits prevent viral entry through their known MOAs with orthogonal compounds or knockdown of putative cell targets. In this manner, the exemplary CNV-G assay can be used to identify novel anti-CHPV chemical probes and molecular targets that modulate CHPV cellular entry.

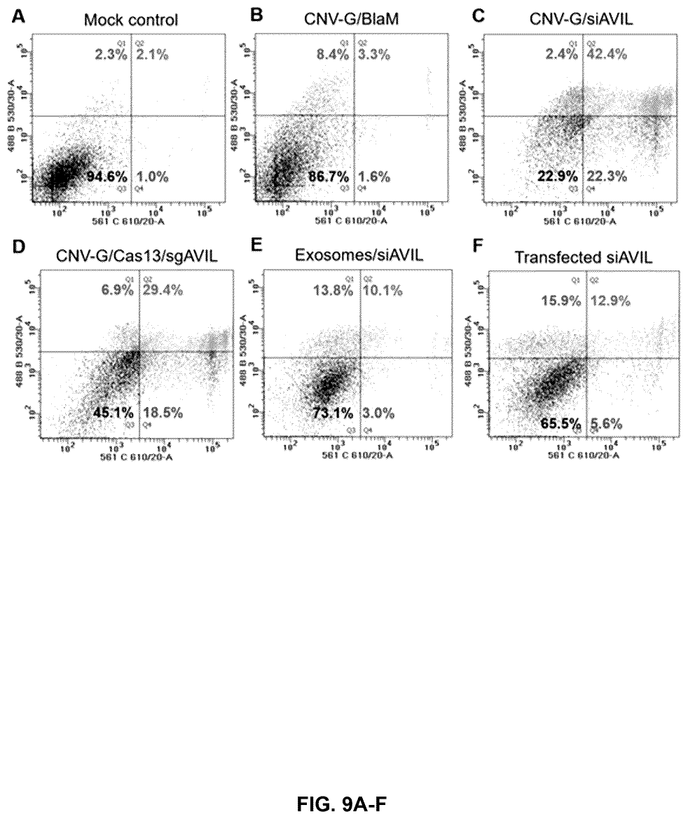

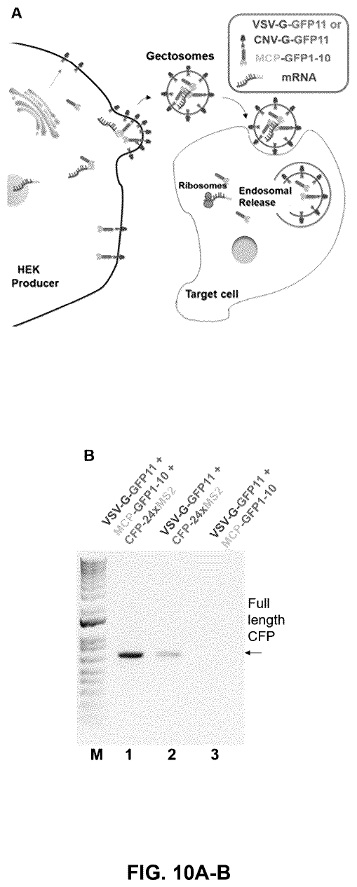

In one specific embodiment, the invention includes systems, methods and compositions to identify small molecules that can block CNV-G gectosome entry and by extension Chandipura viral cell entry. A primary screen and the counter screens can yield a list of compounds with high potency (IC50<5 μM) and low toxicity (IC50>10 μM). Since we have fully confirmed our initial hits as either a viral glycoprotein specific blocker (i.e., CNV-G vs. VSV-G) or pan-inhibitor (e.g., chloroquine), the present inventors can use gectosome and pseudotyped viral infection assay to classify hit compounds. In another embodiment, the present inventors can test whether hits identified through the HTS also block VSV-G gectosome uptake similar to what has been shown in FIG. 9. These studies will allow us to classify hits into viral glycoprotein specific or pan inhibitors. The present inventors may use an exemplary CNV-G or VSV-G pseudotyped lentiviral infection assay to further confirm the specificity of hits as described in FIG. 10. Dose-dependent compound inhibition of viral infection can be performed to obtain IC50 values of the hit compounds for each virus. It is possible that some compounds may have general antiviral activity. To test this activity, it can first be determined if the pan-inhibitors also block Moloney Leukemia Retroviral (MuLV) infection which a retrovirus that is routinely used for stable ectopic expression of reporter genes such as Cre or FRET reporters.

Some of the identified inhibitors are likely to have known cellular targets. For example, Latrunculin B is known to target actin polymerization and U-73122 targets phospholipase C. The present inventors can perform orthogonal validation of hit compounds with known targets. For example, other compounds such as Cytochalasin D or Jasplakinolide, both target actin polymerization albeit through different mechanisms. If these two compounds exhibit similar activity towards CNV-G gectosomes, the inhibition maybe confirmed as a class mechanism rather than a compound-specific mechanism. Independently genetic approaches such as RNAi, CRISPRi, CRISPR KO for target validations can be used.

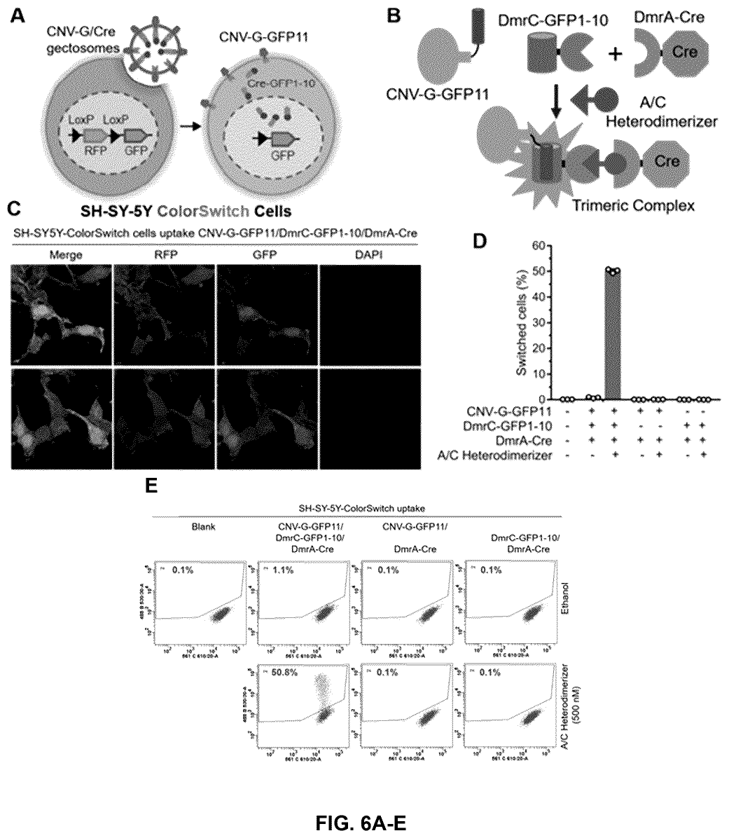

To ensure that the effects hits identified are not restricted to neuroblastoma cell lines in vitro, compound efficacies can be evaluated on primary mouse neuronal and primary human neuronal cells in vitro. As mentioned previously, CHPV is neurotropic and CHPV can infect mice and produce similar neurologic effects seen in children. In FIG. 6, the present inventors demonstrate isolated neural cells using the Adult Brain Dissociation Kit (Miltenyi Biotec) and a gentleMACS™ Dissociator. These cells can be cultured for the short term and allow a determination of the effect of a hit compounds on the uptake of CNV-G gectosomes in different types of neuronal cells. Cells from the adult brain of nTnG mice can be harvested and cultured on poly-L-lysine coated plates. CNV-G/Cre gectosomes can be applied to cultured cells at 10⁴ particles per cell in the presence or absence of hit compounds. Cells may be fixed with paraformaldehyde and stained with antibodies against NeuN (neurons), GFAP (astrocytes) and IBA1 (microglia) to identify the types of cells susceptible to CNV-G gectosome the uptake and the effect of inhibitors on the efficiency of uptake. Primary human astrocytes (ThermoFisher) and primary neurons (Neuromics) are available for purchase. These primary cells may further be used to confirm the results obtained from mouse studies.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference. The terminology used herein is for describing particular embodiments and is not intended to be limiting. As used herein, the singular forms “a,” “and” and “the” include plural referents unless the content and context clearly dictate otherwise. Thus, for example, a reference to “a” or “the” marker may include a combination of two or more such markers. Unless defined otherwise, all scientific and technical terms are to be understood as having the same meaning as commonly used in the art to which they pertain. For the purposes of the present invention, the following terms are defined below.

As used generally herein, a fusiogenic peptide continuing E is generally referred to as “gectosomes.” For example, a VSV-G-containing EV is generally referred to as “gectosomes,” or “VSV-G gectosome.” CNV-G-containing EVs are generally referred to as “gectosomes,” or “CNV-G gectosomes” Cara-G-containing EVs can generally referred to as “gectosomes,” or “Cara-G gectosomes,” COCA-G containing EVs are generally referred to as “gectosomes,” or “COCA-G gectosomes,” MARA-G containing EVs are generally referred to as “gectosomes,” or “MARA-G gectosomes,” MOKV containing EVs are generally referred to as “gectosomes,” or “MOKV-G gectosomes,” NIV-G containing EVs are generally referred to as “gectosomes,” or “NIV-G gectosomes,” RABV-G containing EVs are generally referred to as “gectosomes,” or “RABV-G gectosomes,” CHIKV-G containing EVs are generally referred to as “gectosomes,” or “CHIKV-G gectosomes,” SYN1-G containing EVs are generally referred to as “gectosomes,” or “SYN1-G gectosomes,” SYN2-G containing EVs are generally referred to as “gectosomes,” or “SYN2-G gectosomes,” and HERVK-G containing EVs are generally referred to as “gectosomes,” or “HERVK-G gectosomes.” In other embodiments, EVs containing one or more fusogenic proteins may also be referred to as an “gectosomes.” For example, a SARS-CoV-2 Spike protein containing EV is generally referred to as “gectosomes,” or “S-gectosomes” MERS-G containing EVs are generally referred to as “gectosomes,” or “MERS-G gectosomes,” or M-Gectosome,” all of the foregoing being generally referred to a “gectosome(s) of the invention.”

CNV-G gectosomes may include a G-protein according to SEQ ID NO.'s 1 or 2, or a fragment or variant thereof. Cara-G gectosomes may include a G-protein according to SEQ ID NO.'s 4-6, or a fragment or variant thereof. SARS-G gectosomes may include a G-protein according to SEQ ID NO. 7, or a fragment or variant thereof. VSV-G gectosome may include a G-protein according to SEQ ID NO.'s 10 or 11, or a fragment or variant thereof. RAB-G gectosomes may include a G-protein according to SEQ ID NO. 14, or a fragment or variant thereof. NIV-G gectosomes may include a G-protein according to SEQ ID NO.'s 15 or 16, or a fragment or variant thereof. COCA-G gectosomes may include a G-protein according to SEQ ID NO. 17, or a fragment or variant thereof. MARA-G gectosomes may include a G-protein according to SEQ ID NO. 18, or a fragment or variant thereof. MOKV-G gectosomes may include a G-protein according to SEQ ID NO. 19, or a fragment or variant thereof. CHIKV-G gectosomes may include a G-protein according to SEQ ID NO. 20, or a fragment or variant thereof. MERS-G gectosomes may include a G-protein according to SEQ ID NO. 21, or a fragment or variant thereof. SYN1-G gectosomes may include a G-protein according to SEQ ID NO. 22, or a fragment or variant thereof. SYN2-G gectosomes may include a G-protein according to SEQ ID NO. 23, or a fragment or variant thereof. HERVK-G gectosomes may include a G-protein according to SEQ ID NO. 24, or a fragment or variant thereof, all of the foregoing being generally referred to a “gectosome(s) of the invention.”

As used herein, the term “p6^{Gag}” refer to an HIV protein comprising a viral L domain. p6 also refers to proteins that comprise artificially engineered L domains including, for example, L domains comprising a series of L motifs. An exemplary HIV-p6^{Gag} is SEQ ID NO. 12. The term “Gag protein” or “Gag polypeptide” refers to a polypeptide having Gag activity and preferably comprising an L (or late) domain. Exemplary Gag proteins motif include a motif such as PXXP, PPXY, PXXY, YXXL, RXXPXXP, RPDPTAP, RPLPVAP, RPEPTAP, PTAPPEY, PTAPPEE and/or RPEPTAPPEE. An exemplary HIV-1 Gag protein Typically, an HIV Gag protein comprises a p6^{Gag} protein motif/sequence according to SEQ ID NO. 12.

As used herein, the term “Split Fluorescent Proteins (SFPs)” means a system having are composed of multiple fragments of the eleven anti-parallel outer β-strands and one inner α-strand of a fluorescent protein. Individually the fragments are not fluorescent, but, when complemented, form a functional fluorescent molecule. Typically, the SPF includes a first fragment known as a “SFP detector” that includes nine or ten contiguous β-strands and the α-strand of the fluorescent protein or a circular permutant thereof, and one or two separate fragments known as the “SFP tag(s)” that include the remaining β-strand or strands. Some tripartite SFP systems are known, which include three separate proteins that can form a fluorescent protein. For example, a tripartite split-Green Fluorescent Protein (split-GFP) system can include an SFP detector including GFP β-strands 1-9 (GFP1-9), a first SFP tag including GFP β-strand 10 (GFP10), and a third SFP tag including GFP β-strand 11 (GFP11). The GFP10 and GFP11 tags can be placed on unrelated polypeptide sequences and detected using the GFP1-9 detector.

As used herein, the term “fusogenic” refers to the fusion of the plasma membrane of the microvesicles to the membrane of the target cell. A “fusogenic vesicle” may include a vesicle that incorporates a fusogenic protein. A peptide may be “fusogenic” or a “fusion peptide” is it has a membrane-fusion moiety or domain.

In the context of the present invention “antigen” refers typically to a substance which may be recognized by the immune system, preferably by the adaptive immune system, and is capable of triggering an antigen-specific immune response, e.g., by formation of antibodies and/or antigen-specific T cells as part of an adaptive immune response.

A “vaccine” is typically understood to be a prophylactic or therapeutic material providing at least one epitope of an antigen, preferably an immunogen. In preferred embodiments, the (pharmaceutical) composition or vaccine according to the invention comprises a plurality or more than one of the antigenic peptides displayed on a gectosome composition vaccine as described herein.

The term “pharmaceutically acceptable” or “pharmacologically acceptable” as used herein, refer to molecular entities and compositions that do not produce adverse, allergic, or other untoward reactions when administered to an animal or a human. The term, “pharmaceutically acceptable carrier” as used herein, includes any and all solvents, or a dispersion medium including, but not limited to, water, ethanol, a polyol (for example, glycerol, propylene glycol, and liquid polyethylene glycol, and the like), suitable mixtures thereof, and vegetable oils, coatings, isotonic and absorption delaying agents, liposomes, commercially available cleansers, and the like. Supplementary bioactive ingredients also can be incorporated into such carriers. A “pharmaceutical compositions” comprises a composition of the invention, and preferably a gectosome of the invention and at least one pharmaceutically acceptable carrier.

The term “fusiogenic protein” or “fusion protein” refers to a protein, and preferably a viral protein that can induce the fusion of the plasma membrane derived envelope of the VLP to the membrane of the recipient cell. It is this mechanism that results in entry of the proteinaceous component of the VLP to the cytosol. The envelope glycoproteins of RNA viruses and retroviruses are well known to bind cell receptors and induce this fusion. Accordingly, these proteins are responsible for the infectivity of these viruses. Other examples of fusiogenic proteins include, but are not limited to, influenza haemagglutinin (HA), the respiratory syncytial virus fusion protein (RSVFP), the E proteins of tick borne encephalitis virus (TBEV) and dengue fever virus, the E1 protein of Semliki Forest virus (SFV), the G proteins of rabies virus and vesicular stomatitis virus (VSV) and baculovirus gp64. Functionally equivalent fragments or derivatives of these proteins may also be used. The functionally equivalent fragments or derivatives will retain at least 50%, more preferably at least 75% and most preferably at least 90% of the fusiogenic activity of the wild-type protein.

Particularly preferred is the envelope viral glycoprotein, from neurotropic Chandipura vesiculovirus (CNV-G) (SEQ ID NO. 1, 2), or a peptide fragment or variant thereof. CNV-G has high fusiogenic activity with high specificity to neuronal cells. Without wishing to be being bound by theory, the molecular mechanism of CNV-G-cell surface interaction consists of attachment, followed by a step of membrane fusion between the membrane of the neuronal cell and the viral envelope. This process has been well documented for the influenza virus haemagglutinin and host cell plasma membranes. Additional fusiogenic proteins include: Cara-G according to SEQ ID NO.'s 4-6, or a peptide fragment or variant thereof; SARS-G according to SEQ ID NO. 7, or a peptide fragment or variant thereof; VSV-G according to SEQ ID NO.'s 10 or 11, or a peptide fragment or variant thereof; RAB-G according to SEQ ID NO. 14, or a peptide fragment or variant thereof; NIV-G according to SEQ ID NO.'s 15 or 16, or a peptide fragment or variant thereof; COCA-G according to SEQ ID NO. 17, or a peptide fragment or variant thereof; MARA-G according to SEQ ID NO. 18, or a peptide fragment or variant thereof; MOKV-G according to SEQ ID NO. 19, or a peptide fragment or variant thereof; CHIKV-G according to SEQ ID NO. 20, or a peptide fragment or variant thereof, MERS-G according to SEQ ID NO. 21, or a peptide fragment or variant thereof; SYN1-G according to SEQ ID NO. 22, or a peptide fragment or variant thereof; SYN2-G according to SEQ ID NO. 23, or a peptide fragment or variant thereof; and HERVK-G according to SEQ ID NO. 24, or a peptide fragment or variant thereof, all of the aforementioned being generally referred to as a “fusiogenic(s) protein of the invention.” In specific embodiments, the present invention also relates to an in vitro method for delivering a cellular mRNA of interest into a target cell, and preferably a neuronal or other human cell, by contacting said target cell with an engineered fusogenic secreted vesicles, such as one or more of the gectosomes of the invention, of having a cargo of a cellular mRNA, of other molecule of interest.

As summarized above, embodiments of the invention include methods of introducing a cellular mRNA, or other target molecules, into a target cell through introduction of an engineered fusogenic secreted vesicle, such as a gectosome of the invention. Such methods include contacting the target cell with a engineered gectosome of the invention. As described above, where the engineered fusogenic secreted vesicles may be present in a composition of a population (for example where the number of engineered fusogenic secreted vesicles ranges from 10³ to 10¹⁶, such as 10⁴ to 10¹³, including 10⁴ to 10⁹), under conditions sufficient for the micro-vesicle to fuse with the target cell and deliver the target protein, nucleotide, or molecule contained in the engineered fusogenic secreted vesicles into the cell. Any convenient protocol for contacting the cell with the engineered fusogenic secreted vesicles may be employed. The particular protocol that is employed may vary, e.g., depending on whether the target cell is in vitro or in vivo. For in vitro protocols, target cells may be maintained with donor cells configured to generate engineered fusogenic secreted vesicles and/or isolated engineered fusogenic secreted vesicles in a suitable culture medium under conditions sufficient for the engineered fusogenic secreted vesicles to fuse with the target cells.

As noted above, target molecules to be delivered to a neuronal cell through a gectosome of the invention may include proteins that may further include research proteins which may include proteins whose activity finds use in a research protocol and/or as a therapeutic protocol. As such, research proteins are proteins that are employed in an experimental procedure. The research protein may be any protein that has such utility, where in some instances the research protein is a protein domain that is also provided in research protocols by expressing it in a cell from an encoding vector. Examples of specific types of research proteins include, but are not limited to: transcription modulators of inducible expression systems, members of signal production systems, e.g., enzymes and substrates thereof, hormones, prohormones, proteases, enzyme activity modulators, perturbimers and peptide aptamers, antibodies, modulators of protein-protein interactions, genomic modification proteins, such as CRE recombinase, meganucleases, Zinc-finger nucleases, CRISPR/Cas nucleases, such as Cas 9 or 13, TAL effector nucleases, etc., cellular reprogramming proteins, such as Oct 3/4, Sox2, Klf4, c-Myc, Nanog, Lin-28, etc., and the like.

Target proteins may be diagnostic proteins whose activity finds use in a diagnostic protocol. As such, diagnostic proteins are proteins that are employed in a diagnostic procedure. The target diagnostic protein may be any protein that has such utility. Examples of specific types of diagnostic proteins include but are not limited to: members of signal production systems, e.g., enzymes and substrates thereof, labeled binding members, e.g., labeled antibodies and binding fragments thereof, peptide aptamers and the like.

Target proteins of interest further include therapeutic proteins. Therapeutic proteins of interest include without limitation, hormones and growth and differentiation factors including, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angiopoietin, angiostatin, granulocyte colony stimulating factor (GCSF), erythroproietin (EPO), connective tissue growth factor (CTGF), basic fibroblast growth factor (bFGF), acidic fibroblast growth factor (aFGF), epidermal growth factor (EGF), transforming growth factor alpha. (TGFα), platelet-derived growth factor (PDGF), insulin growth factors I and II (IGF-I and IGF-II), any one of the transforming growth factor β-superfamily, including TGFβ, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase. Target proteins of interest further include, but are not limited to: fibrinolytic proteins, including without limitation, urokinase-type plasminogen activator (u-PA), and tissue plasminogen activator (tpA); procoagulant proteins, such as Factor Vila, Factor VIII, Factor IX and fibrinogen; plasminogen activator inhibitor-1 (PAI-1), von Willebrand factor, Factor V, ADAMTS-13 and plasminogen for use in altering the hemostatic balance at sites of thrombosis; etc. Also, of interest as target proteins are transcription factors such as jun, fos, max, mad, serum response factor (SRF), AP-1, AP2, myb, MyoD, myogenin, ETS-box containing proteins, TFE3, E2F, ATF1, ATF2, ATF3, ATF4, ZF5, NFAT, CREB, HNF4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins. Also of interest as target proteins are carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylase, hepatic phosphorylase, phosphorylase kinase, glycine decarboxylase, H-protein, T-protein, a cystic fibrosis transmembrane regulator (CFTR) sequence, and a dystrophin cDNA sequence. In one embodiment, the target peptide may be a therapeutic peptide, such as brain-derived neurotrophic factor (BDNF), that may have a therapeutic effect on a neuronal cell, or a therapeutic effect against a neuronal cell-related disease such as Parkinson's or Alzheimer's disease and the like.

Further included are methods for improving the efficacy of a disease therapy by administering or introducing to a subject, in vivo or in vitro a therapeutically effective amount of engineered fusogenic secreted vesicles, such as gectosome of the invention, configured to have a therapeutic effect. In this context, the term “effective” or “effective amount” or “therapeutically effective amount” is to be understood broadly to include reducing or alleviating the signs or symptoms of a disease, improving the clinical course of the disease, or reducing any other objective or subjective indicia of the disease.

The term “nucleic acid” as used herein, refers to a polymer of ribonucleotides or deoxyribonucleotides. Typically, “nucleic acid or “nucleic acid agent” polymers occur in either single or double-stranded form but are also known to form structures comprising three or more strands. The term “nucleic acid” also includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Exemplary analogs include, without limitation, phosphorothioates, phosphoramidates, methyl phosphonates, chiral-methyl phosphonates, 2-O-methyl ribonucleotides, and peptide-nucleic acids (PNAs). “DNA”, “RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide. Disclosure of any nucleotide sequence encompasses both the associated RNA, as well as amino acid sequences, and vice versa, as would be easily ascertainable by one of ordinary skill in the art.

The terms “engineered” or “programmable” comprises fusogenic secreted vesicles that have been modified so as to be non-naturally occurring and that may be configured to load and/or deliver target molecules. The gectosomes of the invention are exemplary programmable fusogenic secreted vesicles that may further exhibit cell or tissue specificity.

As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

In another embodiment, the invention provides polynucleotides that have substantial sequence similarity to a target polynucleotide molecule that is described herein. Two polynucleotides have “substantial sequence identity” when there is at least about 70% sequence identity, at least about 80% sequence identity, at least about 90% sequence identity, at least about 95% sequence identity or at least 99% sequence identity between their amino acid sequences or when the polynucleotides are capable of forming a stable duplex with each other under stringent hybridization conditions. Such conditions are well known in the art. As described above with respect to polypeptides, the invention includes polynucleotides that are allelic variants, the result of SNPs, or that in alternative codons to those present in the native materials as inherent in the degeneracy of the genetic code. Moreover, disclosure of a nucleotide sequence encompasses all corresponding amino acid sequences that it could produce during translation. Conversely, disclosure of an amino acid sequence encompasses all corresponding nucleotide sequences, including DNA and RNA, which correspond could give rise to the peptide considering the redundant nature of the genetic code as described herein.

As used herein, an engineered fusogenic secreted vesicles, such as gectosome, is referred to as “isolated” when it has been separated from at least one component with which it is naturally associated.

The term “introducing,” “administered” or “administering”, as used herein, refers to any method of providing a composition of engineered fusogenic secreted vesicles to a patient such that the composition has its intended effect on the patient. In one embodiment, engineered fusogenic secreted vesicles may be introduced to a patient in vivo, while in other alternative embodiments, engineered fusogenic secreted vesicles may be introduced to subject cells in vitro which may then be administered to a patient in vivo.

The term “patient,” or “subject” as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “cell” as used herein, may include a cell or cells in an in vivo system, such as a subject or patient, or an in vitro system, such as a cell-line or cell-based assay.

The term “coupled” may include direct and indirect connections. In one preferred embodiment, it may mean fused, as in a fusion or chimera protein or molecule.

The term “subject” as used herein refers to a vertebrate, preferably a mammal, more preferably a primate, still more preferably a human. Mammals include, without limitation, humans, primates, wild animals, feral animals, farm animals, sports animals, and pets.

The term “protein,” or “peptide,” or “polypeptide” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of poly- or oligonucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g., 5, 6, 7, 8, 9, 10, 11 or 12 or more amino acids long, including each integer up to the full length of the polypeptide). Those skilled in the art are well versed in methods for selecting antigenic fragments of proteins. As used herein the term “variant” refers to a peptide which has a certain identity with a native or reference compound sequence and still maintain one or more of the properties of the parent or starting peptide.

A further embodiment of the invention relates to the use of DNA editing compositions and methods to inhibit, alter, disrupt expression and/or replace one or more target genes. In various embodiments, one or more target genes may be altered through CRISPR/Cas-9, TALAN or Zinc (Zn2+) finger nuclease systems which may be loaded and delivered through engineered fusogenic secreted vesicles to a recipient cell, the sequence and mode of operation are known in the art and previously described in the parent case, U.S. Provisional Application No. 63/318,304, such definitions being incorporated hereby reference. In some embodiments, the target gene of a cell, tissue, organ or organism is altered by a nuclease delivered to the cell via a strategy or method disclosed herein, e.g., CRISPR/cas-9, a TALEN, or a zinc-finger nuclease, or a plurality or combination of such nucleases. In some embodiments, a single- or double-strand break is introduced at a specific site within the genome by the nuclease, resulting in a disruption of the target genomic sequence.

The terms “comprises”, “comprising”, are intended to have the broad meaning ascribed to them in U.S. Patent Law and can mean “includes”, “including” and the like.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1. Generation of Programable Fusogenic Secreted Gectosome Vesicles for Macromolecular Delivery

Biologics, such as therapeutic antibodies, proteins, RNP and siRNA have become increasingly compelling research tools and therapeutic modality. Delivery of these biologics to the intracellular space of target cells is limited by the fact that the plasma and the endosomal membrane is largely impermeable to biologics. Viral-mediated gene transfer for overexpression, RNA interference, and gene editing poses safety concerns for therapeutics. Extracellular vesicles (EVs) are being developed as a delivery system for intracellular therapeutic proteins or nucleic acids because EVs have high biocompatibility and longer circulation life. However, the lack of control of cargo loading for EVs remains a significant concern. As noted herein on aspect of the current invention is to address the delivery problem with programmable EVs in this application. For example, as previously demonstrated in U.S. patent application Ser. No. 17/164,624 (incorporated herein by reference), engineered EVs, known as gectosomes, are trackable and purifiable to be used as a new type of delivery vehicle for proteins, siRNA and RNA/protein complex such as Cas9/sgRNA.

Gectosomes as delivery vehicles. Many viruses have evolved efficient systems for gaining access to the interior of cellular target by hijacking the endocytic pathways of the host cell via endosomal release. Virus encoded glycoproteins such as vesicular stomatitis virus G protein (VSV-G) promote viral particle attachment to the cell membrane, fusion and endosome escape. Recent studies also revealed that VSV-G is capable of stimulating production of EVs containing VSV-G. These vesicles passively encapsulate numerous cellular proteins and promote nonspecific protein transduction intracellularly. The nonspecific packaging property of vesicles have been exploited to deliver Cas9/sgRNA to edit viral DNA or host genes in mammalian cells. However, in all the studies using a mixture of secreted vesicles, it is unclear what percentage of EVs cells produced is VSV-G EVs and the specific activity of these EVs is unknown. Two other studies employed EVs produced by VSV-G fusion proteins to transfer chimeric membrane proteins.

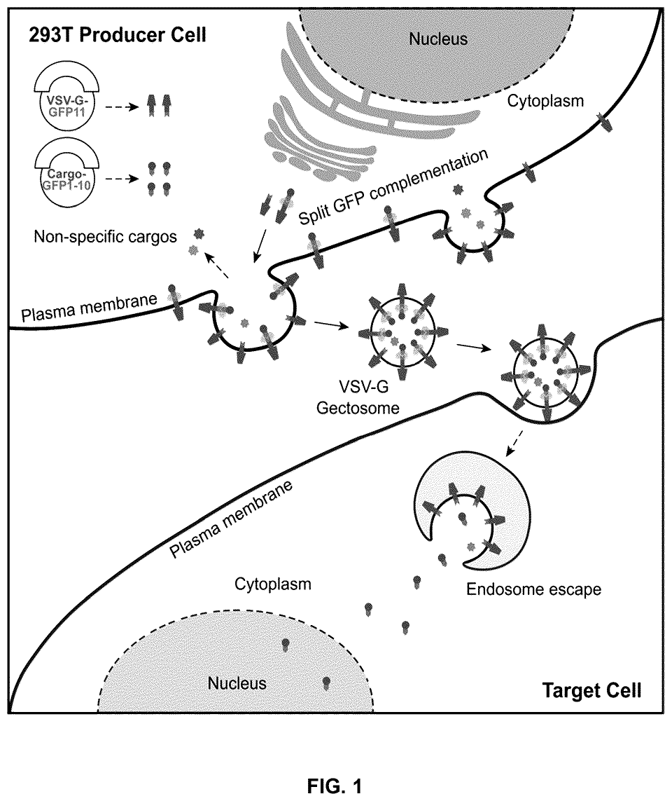

Borrowing from mechanisms of vesicular stomatitis viral delivery and proficient fusogenic activity of vesicular stomatitis virus G protein (VSV-G), as described in co-pending U.S. patent application Ser. No. 17/164,624, (incorporated in its entirety herein by reference), the present inventors engineered a new type of EVs called, gectosomes (G protein ectosomes), which contain two major components: an engineered VSV-G and the cargo of interest tethered to one another via split GFP (FIG. 1). This design is reminiscent of the yeast two-hybrid system. Gectosomes are: (a) genetically encoded and programmable. These gectosomes showed that a variety of cargos can be transferred with the GFP11/GFP1-10 system; (b) because cargo loading reconstitutes GFP fluorescence, gectosomes can be purified quantified based cargo fluorescence; (c) using quantitative proteomics, it was showed that active loading of gectosomes via split GFP outcompetes nonspecific encapsulation of cellular proteins, most notably histones and nucleic acid binding proteins, thereby reduces the heterogeneity of gectosomes; (d) gectosomes can deliver catalytic enzymes, interference RNA, and Cas9 RNPs in a manner not achievable with exosomes; (e) the efficacy of gectosomes delivery is ˜600 fold more efficient than artificial liposomes. When cargo is not tethered to VSV-G, the efficacy is lowered by ˜30 fold. In summary, gectosome technology differentiates itself with the ease of tailoring cargo encapsulation, the reduction in nonspecific cargo transfer, the ability to quantify cargo loading, and purify to homogeneity.

### Example 2. Overexpression of CNV-G in Human Cells Elevates the Production of CNV-G-Containing EVs

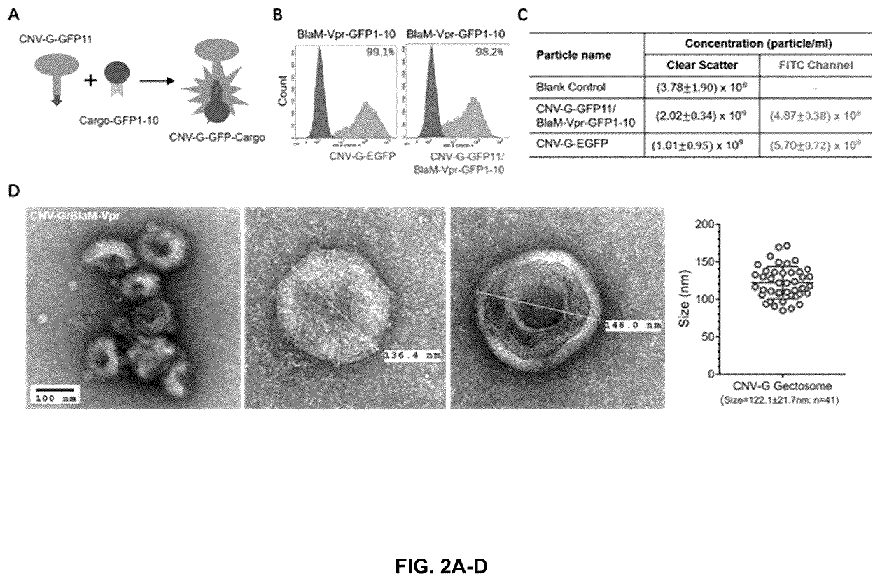

Chandipura virus (CHPV) belongs to the Rhabdoviridae family and is mostly related to the Vesicular Stomatitis Virus (VSV), a virus predominately infects cattle. Unlike VSV, CHPV is an emerging lethal virus to humans. Chandipura virus produces neurological symptoms in naturally infected young children and experimentally infected mice and neuro-tropism is a major feature of this virus. The viral glycoprotein of CHPV known as CNV-G is closely related to VSV-G. The present inventors discovered that the expression of CNV-G protein alone, without any other viral components in 293T cells, can turn these cells into “sprinklers” that secrete billions of CNV-G containing extracellular vesicles. To determine whether CNV-G can produce gectosomes using a split GFP system; 293T cells were transfected with CNV-G-GFP11 and BlaM-Vpr-GFP1-10) (FIG. 2AB). The supernatant was collected and subjected to NanoSight nanoparticle tracking and TEM analysis (FIG. 2CD). Fluorescent gectosomes with an average diameter of ˜122 nm can make up as much as ˜25% of total EVs secreted from the transfected 293T cells. Purified CNV-G gectosomes were analyzed by TEM showing the expected size.

### Example 3. Two Component CNV-G Gectosomes Allow for Selective Cargo Loading in Gectosomes and Robust Intercellular Transfer of Specific Payload

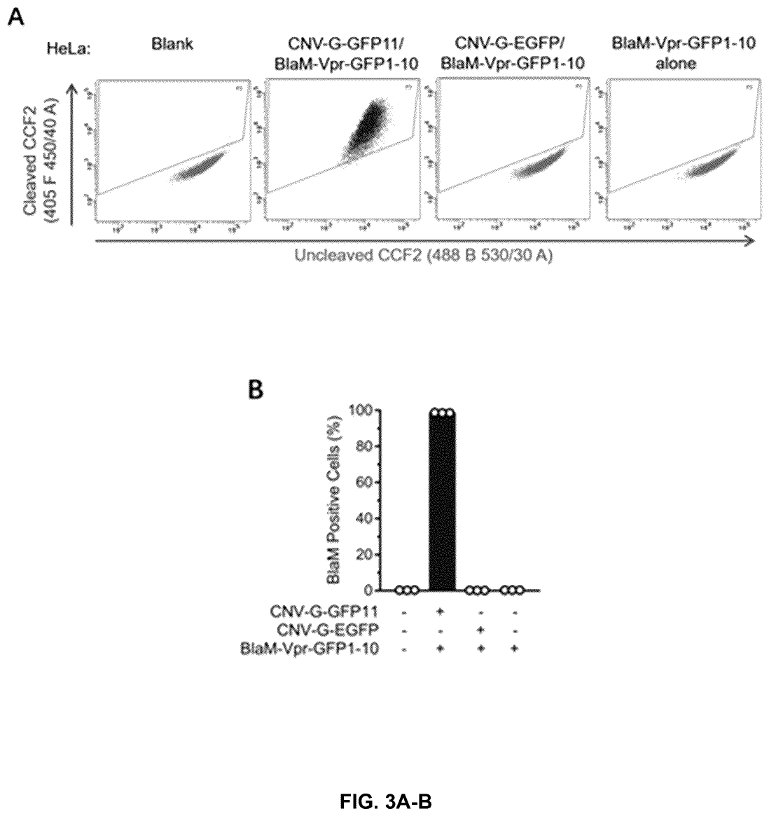

The preset inventors used a split GFP system to construct two-component gectosomes to harness the power of CNV-G to stimulate the production of EVs (FIG. 2A). As shown in FIG. 2, β-lactamase-(BlaM) was fused with GFP1-10 at its N-terminus (GFP1-10-BlaM). The BlaM reporter was selected because its enzymatic activity in cells can be easily measured by flow cytometry using synthetic substrate CCF2-AM, a cell-permeable fluorescent dye composed of 7-hydroxycoumarin-3-carboxamide and fluorescein, bridged by cephalosporin. Upon cleavage of CCF2-AM by β-lactamase, the two fluorophores are separated, causing FRET loss (FIG. 3). In this assay, GFP fluorescence of gectosomes is almost undetectable due to a limited number of particles present in the recipient cells. Additionally, CCF2-AM substrate FITC (488 nm) fluorescence intensity is at least one magnitude higher. CNV-G-GFP11 or CNVG-EGFP with BlaM-GFP1-10 expression vectors were co-transfected into HEK293T cells. While CNV-G-GFP11 can deliver BlaMVpr-GFP11 to HeLa cells, we detect no transfer of BlaM-Vpr-GFP1-10 to recipient cells without its partner or with CNV-G-EGFP (FIG. 3). Importantly, in the case of CNV-G-EGFP, the cargo BlaM-GFP1-10 is not tethered to CNV-G-EGFP. The efficiency of cargo transfer is at least 100 fold less than CNV-G-GFP11 and BlaM-GFP1-10 pair, underscoring the importance of active cargo loading and low specific activity of random cargo loading.

### Example 4. CNV-G Gectosomes have a Narrower Cell and Tissue Tropism

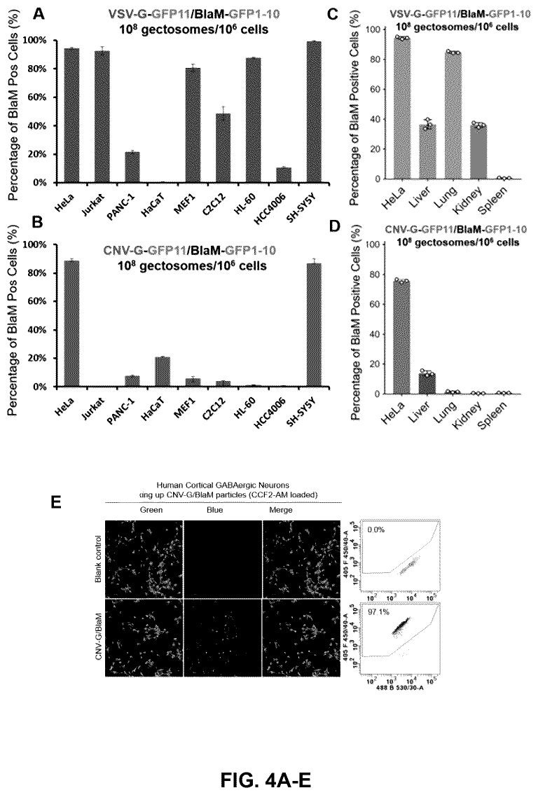

Since Chandipura virus is known for neuro-tropism, cell type specificity of CNV-G-gectosomes uptake was tested. Using the flow cytometry based assay using β-Lactamase (BlaM), VSV-G or CNV-G gectosomes encapsulated with BlaM-GFP1-10 was tested with a number of recipient cell lines. As shown in FIG. 4, VSV-G gectosomes can deliver BloaM to most of cell lines in agreement with the reported pan-tropism of VSV-G. Uptake of CNV-G gectosomes is much more cell-type specific compared to VSV-G gectosomes carrying a BlaM reporter (10⁸ vesicles/10⁶ cells). Besides HeLa cells, only neuroblastoma cell line SH-SY5Y showed substantial uptake. While it is difficult to assess tissue and cell type specificity with cancer cell lines, the fact that most cancer cell lines but neuroblastomas are most susceptible to transduction suggests the selectivity of this viral G protein compared with VSV-G. This result is consistent with the observation that CHPV can breach the blood-brain barrier (BBB) and enter the central nervous system (CNS) to cause encephalitis and neuronal death. To determine the uptake efficiency of VSV-G and CNV-G gectosomes in primary tissues, Applicants isolated primary cells from various tissues of 14-month old BALB/c mice and incubated them with an equivalent number of gectosomes carrying BlaM-Vpr-GFP1-10. HeLa cells were used as a control. As shown in FIG. 4C-D, VSV-G gectosomes can deliver BlaM to cells isolated from the liver, lung and kidney tissues but not the spleen. In contrast, only cells from the liver showed modest uptake of CNV-G gectosomes. The uptake is most likely because the liver cells contain a substantial number of phagocytotic cells (e.g., macrophages). These data show that CNV-G gectosomes are more cell-type and prefer neuroblastoma cells.

To determine whether CNV-G gectosomes can delivery cargo to primary human Cortical GABAergic neurons, CNV-G-GFP1l/BlaM-Vpr-GFP1-10 Gectosomes were incubated with neurons for 16 hours followed by labeling with CCF2-AM and uptake was quantified by fluorescence microscopy or flow cytometry. The results show that CNV-G can mediate robust uptake of BlaM is human primary neurons in culture.

### Example 5. CNV-G Gectosomes Differ from VSV-G Gectosomes in Delivering Cargos that Need to be Released Efficiently to the Nucleus or Cytosol

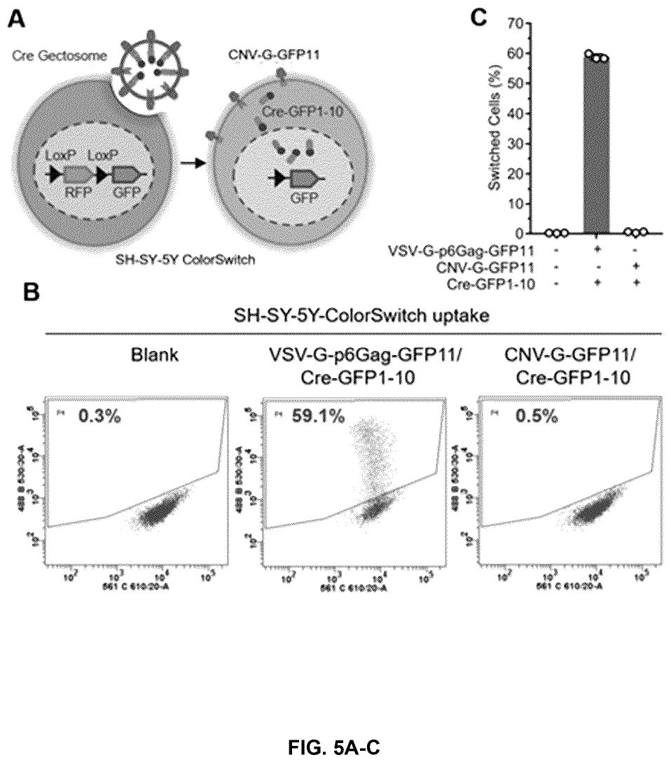

Both BlaM and catalase are enzymes that catalyze reactions with diffusible small molecules, in principle these enzymes should still work as long as gectosomes can internalize to endosomes or even stay in complex with CNV-G. For cargos that act on macromolecules in the cytosol or nucleus, the encapsulated cargo to has to escape from endosomes to reach its target. To test the delivery efficiency of nuclear cargo by CNV-G gectosomes, the present inventors compared the efficacy of Cre-GFP1-10 delivery by VSV-G gectosomes with CNV-G gectosomes. Cre is a recombinase that catalyzes homologous DNA recombination between two loxP sites. We constructed a human neural blastoma SH-SY-5Y reporter cell line stably expressing a tandem DsRed-eGFP cassette. DsRed is terminated by a translation stop codon abound by two loxP sites. In the absence of Cre, eGFP is not expressed due to the stop codon upstream. Upon recombination between the two loxP sites mediated by the presence of Cre, DsRed DNA and along with the stop codon is excised resulting in expression of eGFP and in doing so cells switched from DsRed fluorescence to green fluorescence (FIG. 5A). SH-SY-5Y ColorSwitch cells were incubated with equal number of VSV-G/Cre gectosomes or CNV-G/Cre gectosomes for 48 hr. While large fraction of cells (59%) were switched with the exposure of VSV-G/Cre gectosomes, hardly any cells (<0.5%) changed color with CNV-G/Cre gectosomes (FIG. 5B-C). This result suggest that CNV-G cannot deliver the same cargo in the same manner as VSV-G and may have a different mechanism in cargo delivery and release.

The present inventors hypothesized that the cargo Cre-GFP1-10 may stay bound with CNV-G and does not get released efficiently when it enters the cell. To test this hypothesis, we took the advantage of a chemical inducible hetero-dimerization system commercialized by Takara Bio USA for cargo loading. DmrA (FKBP12) forms heterodimer with DmrC (an 89 amino peptide from mTOR) in the presence of small molecule ligand AP21967. We therefore designed a three component system consisting of CNV-G-GFP11, DmrC-GFP1-10 and Cre-DmrA (FIG. 6B). CNV-G gectosomes with all three or two of the three components were prepared in the presence or absence of AP21967. Robust switch of the reporter cell line was observed only when all three components were present with the addition of AP21967 (FIG. 6D-E). Thus, this result suggest that robust delivery of nuclear cargo using CNV-G gectosomes requires an inducible package or release system. This experiment suggests that CNV-G gectosomes can deliver Cre-GFP1-10 efficiently to neuroblastoma cells with the three-way system.

### Example 6. CNV-G Gectosomes can Deliver Cre Payload to Mouse Brain Tissues In Vitro

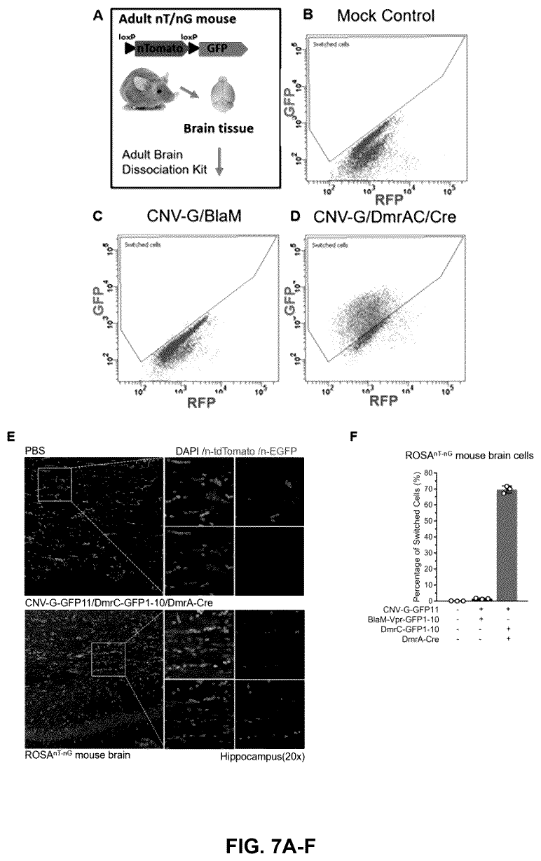

C57BL/6J ROSAnT/nG mice from Jackson Laboratories (Stock No. 023537) harbor a cell nuclear-targeted, two-color fluorescent Cre reporter allele (tdTomato to GFP switch with Cre). A commercialized method (Miltenyi Biotec) to dissociate adult mice brain tissue into single-cell suspensions with the gentleMACS™ Dissociator with Heaters for the mechanical dissociation steps during the on-instrument enzyme incubation was used to isolate somatic neural cells from brain tissue of nTnG mice. Three component CNV-G gectosomes encapsulated with Cre were incubated with cells from dissociated mouse brain overnight prior to flow cytometry analysis. Robust color switch (>80%) was observed with Cre gectosome but NOT the control BlaM gectosome treated cells, suggesting that CNV-G gectosomes can deliver Cre to primary neural cells from mice (FIG. 7).

### Example 7. CNV-G Gectosomes can Deliver Cre Payload to Mouse Hippocampal Tissues In Vivo

To determine whether CNV-G gectosomes can deliver Cre to the brain in live animal, the inventor performed intrahippocampal injection of the CNV-G gectosomes carrying Cre and sectioned the brain issues 14 days following injection. As shown in FIG. 7E-F, >65% nuclei of neuronal cells in the injected area fluoresced green indicating that Cre was successfully delivered into the nucleus of these cells.

### Example 8. CNV-G Gectosome Delivery of Catalase can Suppress ROS-Induced Apoptosis

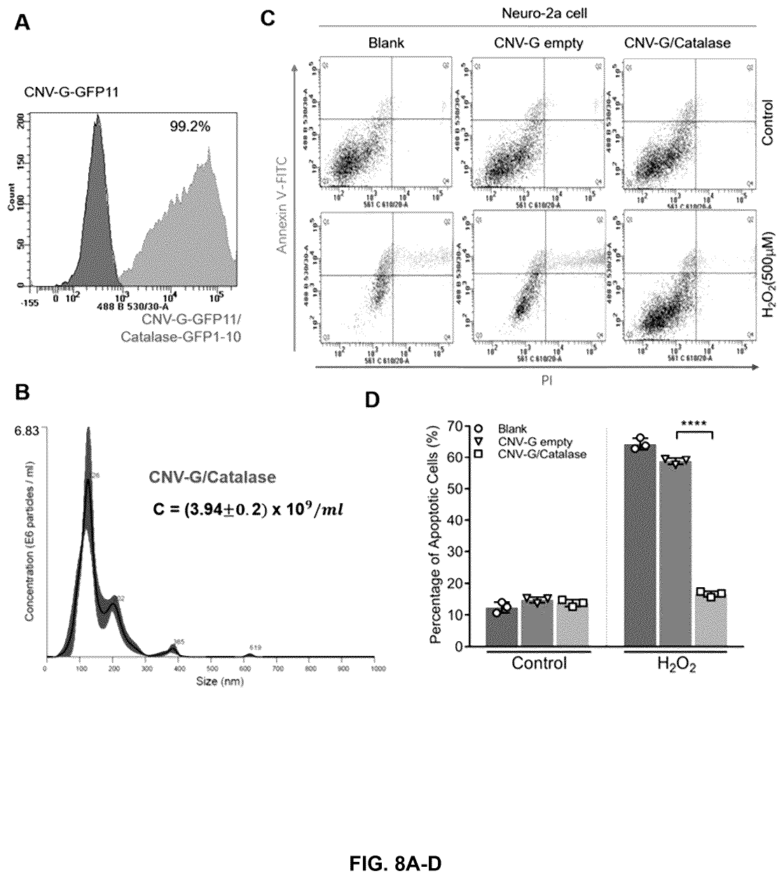

Oxidative stress plays an important role in the degeneration of dopaminergic neurons in Parkinson's disease (PD) and elevated ROS production contributes to neuronal cell death and ultimately neurodegeneration and degradation of motor function. Catalase is one of the most efficient enzymes that removes ROS and has been shown to have neuroprotective effects in a mouse model of PD upon being loaded into exosomes ex vivo. Two component CNV-G gectosomes were prepared by transfecting 293T cells with CNV-G-GFP11 and Catalase-GFP1-10 (FIG. 8A). Efficient production of CNV-G gectosomes carrying catalase (exemplary SEQ ID NO. 8) was observed (FIG. 8B). To determine whether CNV-G gectosomes encapsulated with catalase can protect neuronal cells from peroxide induced cell death in vitro, we incubated Neuro-2A cells with CNV-G gectosomes with catalase or without cargo before exposing cells to peroxide. As shown in FIG. 8C-D, cells that were exposed to CNV-G gectosomes with catalase are resistant to cell death compared with cells treated with CNV-G gectosomes without specific cargos. The percentage of apoptotic cells is determined by flow cytometric analysis of cells stained AnnexinV/PI. Similar observation was made with human neuroblastoma cell line SH-SY-5Y and HeLa cells. These results suggest that CNV-G gectosomes loaded with catalase may provide significant neuroprotective effects in vitro.

### Example 9. CNV-G Gectosome Delivery of siRNA and Cas13a/sgRNA RNP

We showed VSV-G gectosomes can deliver siRNA, shRNA, and SaCas9/sgRNA to recipient cells to alter target gene expression in vitro and in vivo. To determine the potential application of CNV-G gectosomes in targeted therapies in the CNS system, we tested the efficiency of RNA depletion of AVIL, an oncogenic driver protein overexpressed in glioblastoma (GBM), using CNV-G gectosomes in comparison with other delivery methods. Knockdown expression of AVIL in U87 GBM cells is known to trigger cell apoptosis. We collected CNV-G gectosomes loaded with BlaM, or Cre or LwaCas13a/sgAVIL from 293T cells transfected with the expression vectors. Applicants also collected exosomes from untransfected 293T cells. Exosomes and CNV-G/Cre gectosomes were loaded with AVIL siRNA (ThermoFisher) using Exo-Fect™(SBI). U87 cells were incubated with CNV-G/BlaM, or CNV-G/Cre/siAVIL or CNV-G/Cas13a/sgAVIL gectosomes or exosomes/siAVIL for 90 hr. As a control, we also transfected the same amount of siAVIL directly with Lipofectamine™ RNAiMAX. Following incubation, the percentage of apoptotic cells was determined by flow cytometric analysis of cells stained AnnexinV/PI (Biolegend). As shown in FIG. 9, CNV-G gectosomes loaded with siAVIL or LwaCas13a with Crispr AVIL guide RNA caused more cell apoptosis than exosomes or lipid transfection. These results suggest that CNV-G gectosomes can deliver potential therapeutic nucleic acids or RNP to glioblastomas.

### Example 10. CNV-G Gectosomes can Encapsulate Full-Length mRNA

Intercellular mRNA transduction in a cell type-specific manner is a new frontier for developing mRNA therapeutics. mRNAs are more desirable to transduce compared to proteins since the potential amplification of effects of the material transferred without risking permanent genome changes. Using RT-PCR, the Applicants shows full length CFP with 24 copies of MS2 stem loop sequence at the 3′-UTR can be detected CNV-G gectosomes when CNV-G gectosomes encapsulate MCP which is a RNA binding protein that bind MS2 specifically. Results in FIG. 10 shows that it is possible to encapsulate full-length mRNA in gectosomes using a strategy outlined in FIG. 10A.

### Example 11. Co-Encapsulation of CD47 with CNV-G Gectosomes Suppresses CNV-G Gectosome Clearance by Macrophages

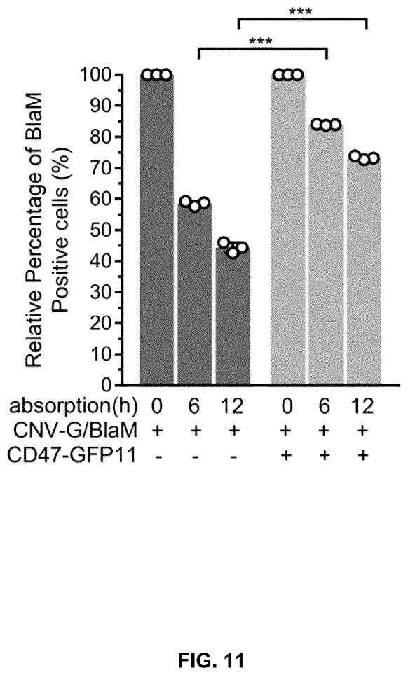

Circulating monocytes, macrophages, dendritic cells, and neutrophils remove dead cells, cell debris, exosomes, and ectosomes through phagocytosis. These phagocytic cells express signal regulatory protein α (SIRPα), which serves as a receptor for CD47, a transmembrane protein present in high levels in tumor cells and normal cells alike. Binding of CD47 to SIRPα triggers a “don't eat me” signal. Previous studies showed that the presence of CD47 (SEQ ID NO. 9) on exosomes suppresses their depletion by phagocytosis, resulting in higher exosome levels in the blood. To test if the CD47-SIRPα system plays a role in CNV-G gectosome clearance by macrophages in vitro, the present inventors overexpressed Myc and GFP11-tagged mouse CD47 in 293T cells, along with the standard CNV-G gectosome components (CNV-G-GFP11/BlaM-GFP1-10). Next, we incubated control and CD47 gectosomes containing BlaM-GFP1-10 with mouse RAW 264.7 macrophages for 6 or 12 h. The supernatants were recovered after incubation, and BlaM activity assays measured the amount of Gectosomes remaining in the media. RAW 264.7 cells depleted approximately 40% and 60% of the control Gectosomes after 6 h and 12 h (FIG. 11). In contrast, only 20% and 30% of CD47 gectosomes were depleted. These results suggest that displaying CD47 on the surface of CNV-G gectosome can slow down depletion of gectosome clearance by macrophages.

### Example 12. CNV-G Gectosomes Differ from VSV-G Gectosomes in Pharmacokinetics and Biodistribution

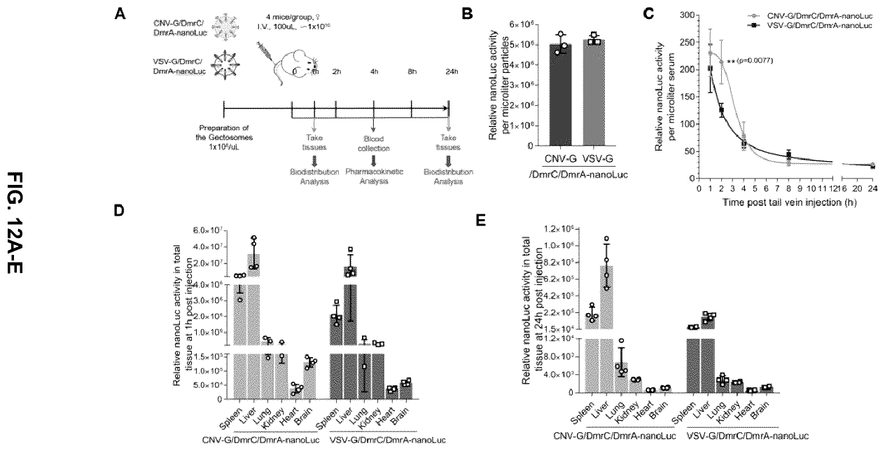

The present inventor sought to evaluate the ability of different gectosomes to target certain cells as well as their individualized pharmacokinetics. As shown in FIG. 12A-B, four (4) week old mice were prepared and injected with preparations (3×10⁸/uL) of CNV-G-nanoLuc and VSV-G-nanoLuc gectosomes. Injection was in the mouse tail and tissues samples were taken at 1-hour and nanoLuc signal activity was evaluated to determine the cell targeting characteristics of CNV-G and VSV-G gectosomes respectably. Notably, all RLUs were obtained under the same total protein amount (45 ug/test) and are normalized to per microgram of tissue lysates, and the activity values are normalized to the same nanoLuc activity injected initially.

As shown in FIGS. 12D-E, biodistribution data indicates that CNV-G was differentially localized to certain tissue compared to VSV-G. In some cases between a 1× to 4× fold between CNV-G and VSV-G was observed. The present inventor next sought to evaluate the ability of different gectosomes to present individualized pharmacokinetics. As shown in FIG. 12C, mice were prepared and injected with preparations (3×10⁸/uL) of CNV-G-nanoLuc and VSV-G-nanoLuc gectosomes. Injection was in the mouse tail and blood samples were taken over a 24-hour time-course. The nanoLuc signal activity was evaluated to determine the pharmacokinetics characteristics of CNV-G and VSV-G gectosomes in the blood sample respectably. As shown in FIG. 12C, CNV-G and VSV-G presented different activity in the samples blood plasma over the time-course, with CNV-G exhibiting a higher-level of activity over time based on RLU's per milliliter.

### Example 13. CNV-G Gectosomes Differ from VSV-G Gectosomes Enter Cells Using Different Cell Surface Receptors

Differences in cellular tropism and biodistribution between CNV-G and VSV-G gectosomes could be a result of distinct cellular receptors they utilize to gain entrance into the cells. The cellular entry receptor for VSV-G is known to be LDLR. To test whether CNV-G gectosomes utilize the same receptor to enter into the same cells, Applicants performed knockdown of LDLR in 293T ColorSwitch cells. As shown in FIG. 13, transfecting siRNA into 293T with siRNA specifically targeting LDLR results in a lowered expression of this receptor by real time qPCR analysis. As expected, 293T ColorSwitch cells with the LDLR knockdown exhibit poor uptake of VSV-G gectosome mediated Cre delivery. However, LDLR knockdown does not affect CNV-G gectosome delivery of Cre. These results suggest that CNV-G and VSV-G uses different cell surface receptors to enter the cells, which is a basis for their different tropism.

### Example 14. Other Viral Glycoproteins and Human Endogenous Envelope Proteins that can Promote Gectosome Production

Vesicular stomatitis virus belongs to the Rhabdoviridae family that includes 18 genera and 134 species of viruses. Like VSV-G, viruses in this family use envelope glycoprotein for attachment to the host cell surface and for mediating viral entry. However, different viral envelope glycoproteins have been evolved to use different cellular receptors as the port of entry. Accordingly, the tropism of Rhabdoviridae family is highly diverse. Additionally, other family of viral glycoproteins are also known to have distinct tropism as a result of receptor usage. The inventor screened more than 30 viral glycoproteins for their ability to promote production of EVs. Several of these viral glycoproteins including CARA-G, NIV-G, COCA-G, MARA-G, MOKV-G, CHIKV-G, S, SM, SYN1-G, SYN2-G, and HERVK-G were proficient in stimulating gectosomes production (Table 1 and 2). As another example of gectosome delivery of cargo using specific viral glycoprotein, Cara-G can deliver BlaM-GFP1-10 to several cell lines very efficiently including PANC-1 and HaCaT which are refractory to CNV-G gectosomes or poorly respond to VSV-G gectosomes (FIG. 4AB). Thus, different gectosomes decorated by different types of viral glycoproteins offer a means to target specific cell type or tissues.

### Example 15. Side-Step Adaptive Immunity with Alternate Dosing of Different Types of Gectosomes

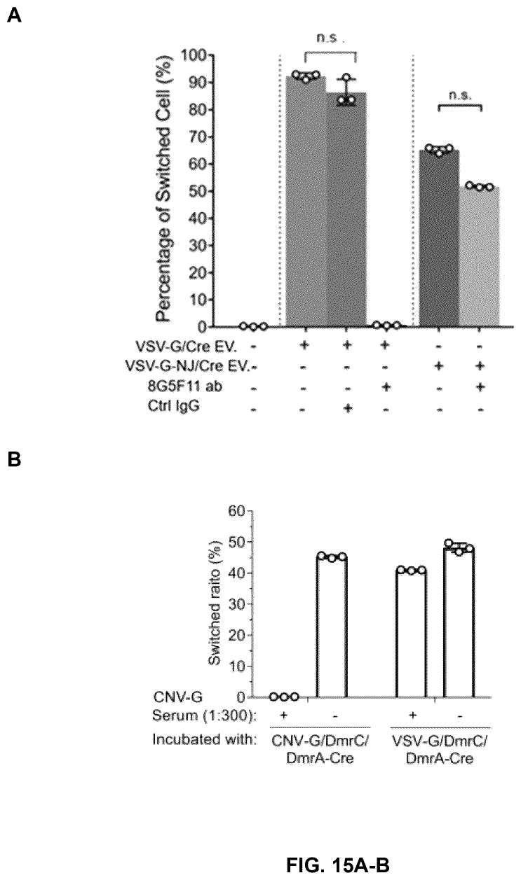

A major limitation for virus-based gene delivery lies in the elicitation of host-immune response due to persistent expression of viral proteins. One way to address this potential issue is to employ different subtypes of VSV-G to sidestep the adaptive immunity through alternate dosing of different pseudotyped gectosomes. To demonstrate the feasibility of this approach, the present inventors tested whether a neutralizing antibody (8G5F11) against VSV-G exerts the same effect on a different VSV-G subtype. The VSV-G used throughout prior studies is from the Indiana strain (SEQ ID NO. 13). VSV-G-NJ, a variant from VSV New Jersey strain (SEQ ID NO. 10, 11) that shares ˜50% homology to VSV-G Indiana, can promote gectosome production and mediate protein (e.g., Cre) transduction just like VSV-G. As shown in FIG. 15A, 8G5F11 blocks VSV-G gectosome delivery of Cre-GFP1-10 but has no effect on that of VSV-G-NJ. This result suggests that VSV-G New Jersey variant can be used for alternate dosing because immunity developed for VSV-G Indiana does not affect delivery with VSV-G New Jersey. Similar experiment was performed with CNV-G vs VSV-G gectosomes carrying Cre in a three-way system as FIG. 6. Neutralizing antiserum against CNV-G only block CNV-G gectosomes uptake in mouse N2A neuroblastoma cells but not VSV-G gectosomes (FIG. 15B). Collectively, these results suggest that it is possible to sidestep host adaptive immune response through alternate dosing of different type of gectosomes.

### Example 16. Gectosomes Provide a High-Throughput Screening Platform for Identifying Anti-Viral Entry Inhibitors

All enveloped viruses encode one or more viral surface glycoproteins to facilitate viral binding to the cell surface, virus-cell fusion, viral particle intracellular release or virus spreading through pathological cell-cell fusion. Since various gectosomes can deliver Cre to reporter cells to switch their genotypes and resulting phenotypes, robust assay for viral glycoprotein-dependent cell entry can be developed and used for screening inhibitors that can block gectosomes uptake, a proxy for viral particle cellular entry. The present inventors, in one aspect of the invention describe methods and compositions small molecule CHPV entry inhibitors through a screening methodology.

To develop a high throughput screening (HTS) assay to identify CNV-G cell entry inhibitors, the present inventors adapted the system described in FIG. 5 into a cell-based screen platform. Since the uptake of CNV-G gectosomes carrying Cre by the reporter cell line will result in fluorescence switch from red to green, the ratio of GFP/RFP can be readily quantified from scanned images acquired by PerkinElmer Phenix Opera HTS microscope. Briefly, images were flat-field corrected, then nuclear and cellular regions were determined using the Hoechst 33342 and RFP channels, respectively. Cells without a baseline RFP expression were excluded. Next, cellular regions were measured for mean GFP expression. Cells above a discernable threshold were counted as GFP-positive. Finally, the count of GFP-positive over RFP-positive cells was calculated as a percent for each well. Mean, SD, Z′ and graphs were generated using MATLAB scripts we have published. Hoechst 33342 live-cell DNA dye was added for cell identification, quantitation and assessment of nuclear integrity, an indicator of cell health and viability.

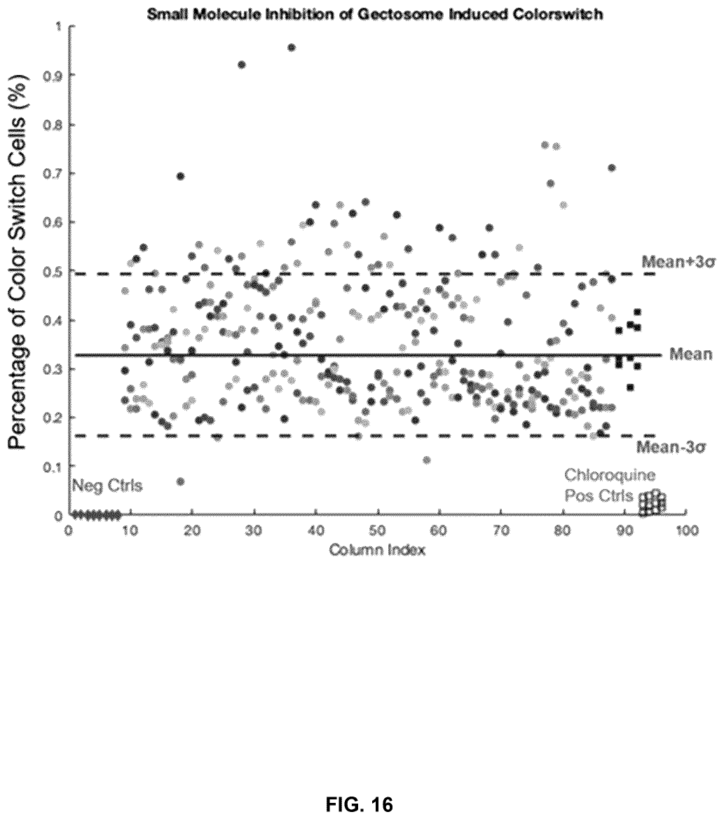

The present inventors optimized HTS screen assays with a 96-well CNV-G gectosome Cre delivery study to determine the robustness and reproducibility of our assay. The uptake assay exhibited a robust assay signal window with a signal/background (S:B) ratio of >20 fold and a Z-factor coefficient of 0.65. Next, we performed a small pilot screen of 320 compounds selected from NCI/DTP Diversity Set with chloroquine as positive controls. The Z′ factor coefficients of the four plates is ≥0.5. Three hits with a Z score <−3 were found to inhibit CNV-G gectosome induced color switch (FIG. 16). Dozens of wells in which Z score >3 were also found to enhance CNV-G gectosome uptake. Further examination of these wells indicated that some of them are colored compounds and most likely represent assay interference compounds. Highly cytotoxic compounds tend to produce autofluorescence signals in the green channel. Since our major focus is to discover viral entry inhibitors, they were not subjected to follow-up analysis at this stage of the investigation.

### Example 17. Pilot HTS Hits Confirmation and Validation

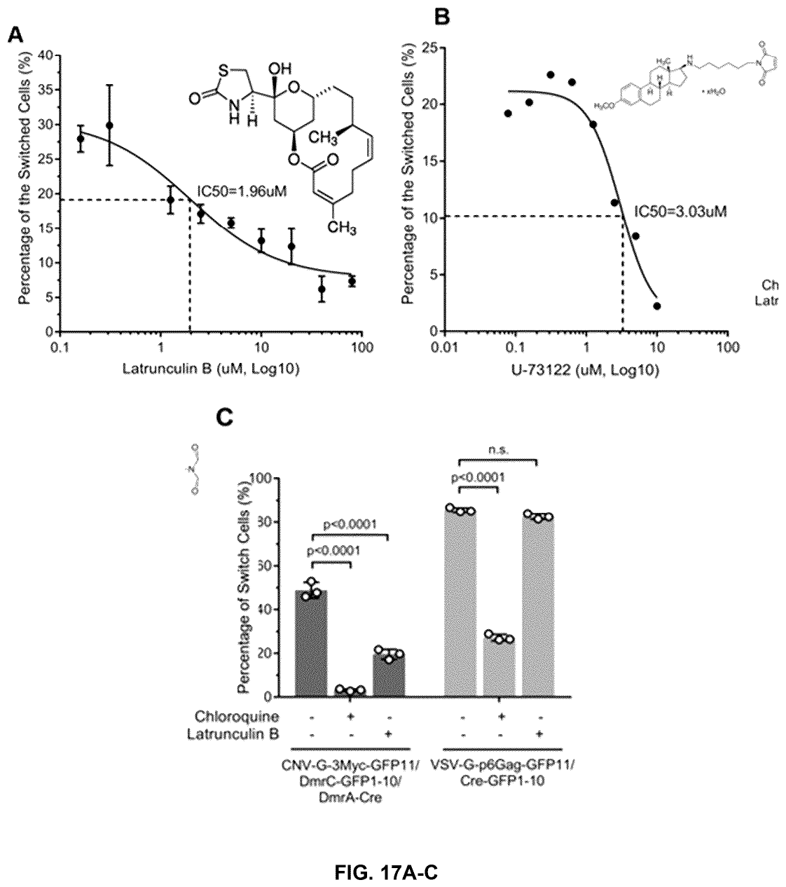

Four hits were first evaluated for efficiency and toxicity. NSC622608 and NSC36758 exhibited significant cytotoxicity. Since we are interested in potent antiviral entry inhibitors, cytotoxic compounds with nM to sub μM cell killing are not particularly appealing. The two hits that did not kill cells from the screen are Latrunculin B and U-73122. Latrunculin B is a G-actin sequester that prevents F-actin assembly and. Surprisingly, Latrunculin B is not as toxic at the concentration (>90% cell viability) we used as it is known to be gradually inactivated by serum and not as cytotoxic as Latrunculin A. U-73122 is a phospholipase C inhibitor that blocks agonist-induced platelet aggregation and neuropathic pain. Latrunculin B and U-73122 inhibit CNV-G gectosome-mediated Cre transfer (IC50=1.96 μM and IC50=3.03 μM) (FIG. 17A). The specificity of Latrunculin B and U-73122 as hits for CNV-G gectosomes was evaluated by investigating its activity against VSV-G gectosomes. Unexpectedly, Latrunculin B does not inhibit VSV-G gectosome-mediated Cre delivery under the same conditions while U-73122 inhibits both (not shown). An independent flow cytometry assay was used to confirm that while chloroquine inhibits both CNV-G and VSV-G gectosome cellular uptake and Cre cargo release, Latrunculin B only inhibits CNV-G and has virtually no effect on VSV-G (FIG. 17B). This result suggests that our screen and hit confirm assays can identify viral glycoprotein specific or pan inhibitor hits without significant cytotoxicity. The effect of Latrunculin B on CNV-G but not VSV-G was further validated using a VSV-G or CNV-G pseudotyped lentiviral infection assay further confirm the authenticity of the hits.

### Example 18. Identification Anti-CHPV Compounds that Selectively Block the CNV-G Function

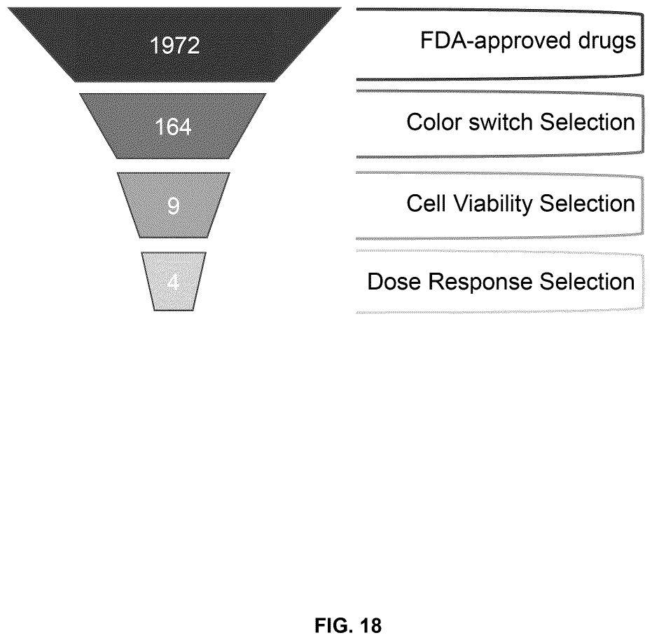

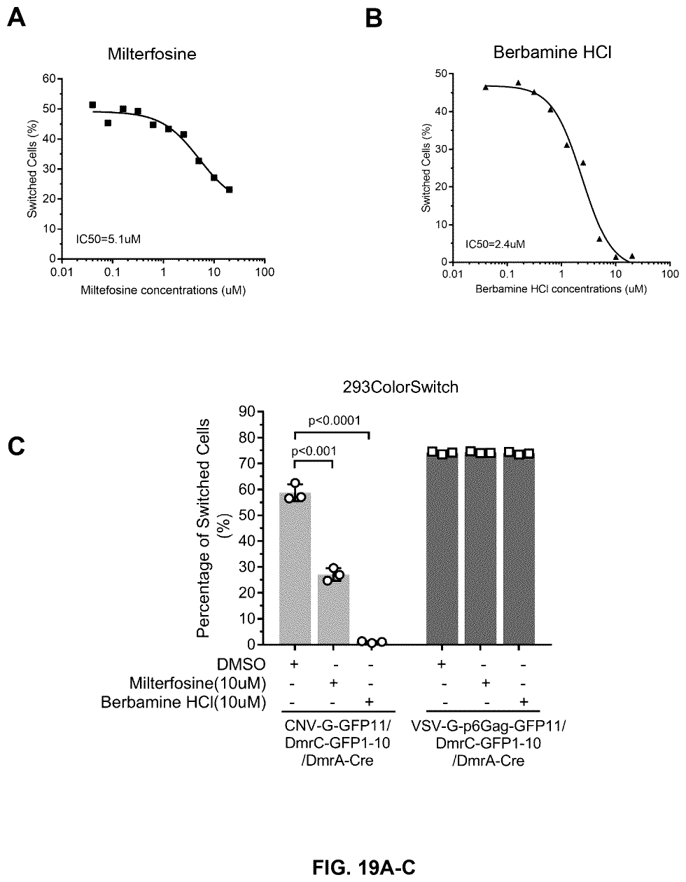

The feasibility of identifying CNV-G gectosome specific entry inhibitors was shown with a proof-of-concept pilot screen using a library of FDA approved drug. FIG. 18 shows the screen funnel for hit triage. Two hits from the screen were further tested by dose-responsive analysis and viral glycoprotein specific inhibition assay using CNV-G or VSV-G gectosomes. Results show that Milterfosine and Berbamine HCl specifically inhibit CNV-G gectosome uptake but NOT VSV-G gectosome uptake (FIG. 19), suggesting that gectosome based HTS can identify small molecules that inhibit viral glycoprotein mediated cellular entry.

### Example 19. Induction of Host Adaptive Immune Responses and Antibody Production Against Viral Glycoprotein or Payloads with Gectosomes

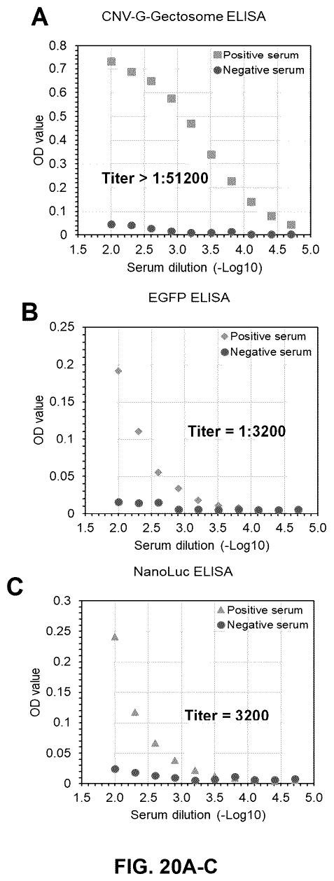

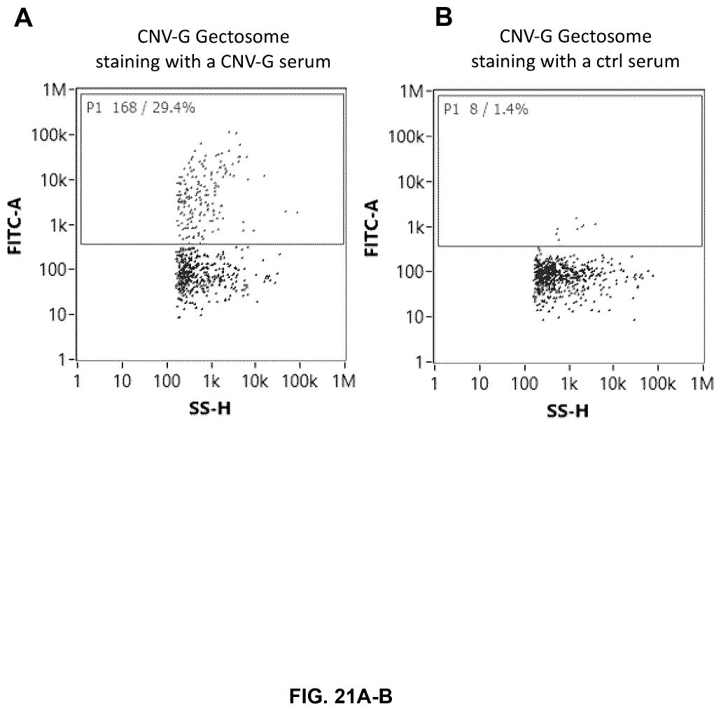

As noted above, in certain embodiments gectosomes could provide a means to induce host adaptive immune response and thereby can be used for vaccination against viral infection or mounting antitumor response if tumor neoantigens are encapsulated. To demonstrate the feasibility of this application, the inventors dosed CNV-G-GFP11 gectosomes encapsulated with Nano-Luc-GFP1-10 via intravenous or intramuscular administration. Five injections with 2×10E¹⁰ gectosomes each time were administered every three weeks. Sera were collected every two weeks after each injection and tested for antibody activity using ELISA. Recombinant GFP (Origene) or Nanoluc (Promega) or CNV-G gectosomes were used for quantifying titers. Serum from naïve animals were used as a control. After the third injection, sera showed positive reactions to all three antigens with the highest titer for CNV-G (FIG. 20). Neutralizing antibody specifically targeting CNV-G was produced in the injected animals based on the functional neutralization assay (FIG. 15). The specificity of CNV-G antisera was independently confirmed by a vesicle flow cytometry assay using a Laser Flow NanoAnalyzer (NanoFCM). The CNV-G gectosomes was stained with an anti-CNV-G or control antiserum showing ˜30% of EVs are positive for CNV-G (FIG. 21). Thus, gectosomes can induce antibody production to both the viral glycoprotein and the encapsulated cargos.

### Example 20: Reducing Immunogenicity of Gectosomes with Non-Immunogenic GFP Variant or Low Immunogenic Chemical-Induced Dimerization Nanobodies

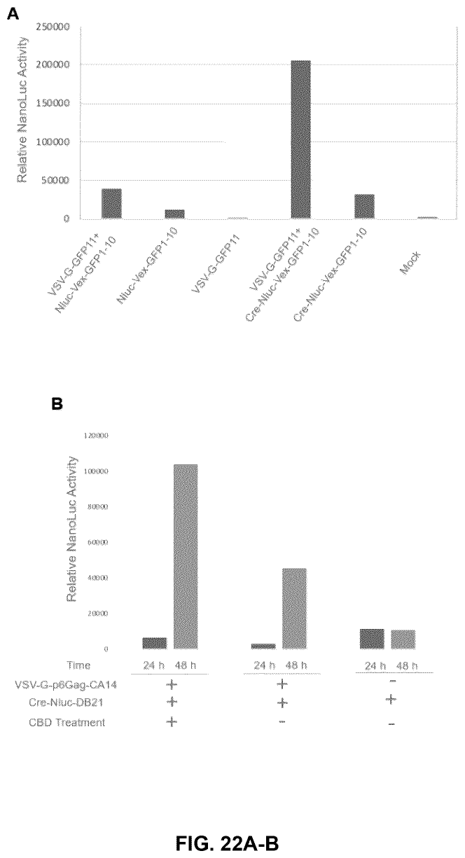

In certain embodiments, it is highly desirable to minimize host immune responses to the module (e.g. split GFP) that mediates the dimerization of the viral glycoprotein with the encapsulated cargos. While GFP is immunogenic, previous studies show that a variant of GFP known as Vex-GFP (violet-excited GFP) does not induce immune response (LaFleur et al. 2019). The inventors test whether Vex-GFP can be split in the same way as GFP and promotes gectosome production in a manner similar to GFP. As shown in FIG. 22A, gectosomes collected from 293T cells co-expressing VSV-G-GFP11 with either Nluc-Vex-GFP1-10 or mScarlet-Nluc-Vex-GFP1-10 but not individually contain robust activity of encapsulated Nluc. As in the case of split GFP, coexpression of both partners reconstitutes blue fluorescence. Thus, Vex-GFP1-10 (SEQ. ID NO. 25) can substitute GFP1-10 in applications where low immunogenicity of cargo is desired.

Nanobodies, derived from heavy chain-only antibodies in camelids, are known to possess a low immunogenicity risk profile (Ackaert et al. 2021). A dual-nanobody cannabidiol (CBD) sensor which consists of two nanobodies that would only heterodimerize in the presence of the ligand has been developed (Kang et al. 2019). The inventors tested whether CBD inducible dimerization of nanobody CA14 (SEQ ID NO. 26) and DB21 (SEQ.ID NO. 27) can be used as an active loading mechanism similar to the DmrA/DmrC system. VSV-G-p6Gag-CA14 and Cre-Nluc-DB21 were coexpressed or alone in 293T cells in the presence or absence of CBD treatment. Gectosomes were collected from transfected cells and Nluc activities were determined. CBD induces an elevated production of gectosomes encapsulated Nluc cargo after 48 hr (FIG. 22B). Therefore, split Vex-GFP or CA14/DB21/CBD provides means for active loading of gectosomes while lowering the risk of immunogenicity for certain embodiments.

### Example 21: Incorporated Embodiments

The compositions and methods described in originally numbered claims 16-67, 80-84, and 89-93, described in the parent case, U.S. Provisional Application No. 63/318,304, are specifically incorporated hereby reference.

TABLE 1
Exemplary gectosomes incorporating unique viral glycoprotein.

| | | | Particle | Particle |
|---|---|---|---|---|
| Viral Glycoprotein | | GFP signal | concentration | size |
| source | Gectosomes Configuration | in 293T | (particles/mL) | (nm) |
| Cocal virus | Coca-G-GFP11/BlaM-Vpr-GFP1-10 | ✓ | 7.4E+07 | 173.7 |
| Maraba virus | Mara-G-GFP11/BlaM-Vpr-GFP1-10 | ✓ | 9.7E+07 | 126.1 |
| Ebola virus (MOKV) | EBOV wt-GFP11/Cre-GFP1-10 | ✓ | 5.6E+08 | 303.2 |
| Nipah virus (NiV) | NiV-G/Niv-F-GFP11/Cre-GFP1-10 | ✓ | 5.6E+07 | 268.8 |
| Rabies virus (RabV) | Rabies-GFP11/Cre-GFP1-10 | ✓ | 2.2E+09 | 201.6 |
| Chikungunya virus | CHIKV-E3E2E1-GFP11/Cre-GFP1-10 | ✓ | 5.3E+07 | 189.0 |
| (CHIKV) | | | | |
| Human coronavirus | SARS-S-GFP11/Cre-GFP1-10 | ✓ | 1.4E+08 | 161.8 |
| (SARS-CoV2) | | | | |
| Human beta coronavirus | MERS-S-GFP11/BlaM-Vpr-GPP1-10 | ✓ | 6.0E+07 | 146.0 |
| (MERS-CoV) | | | | |
| Carajas virus | Cara-G-GFP11/Cre-GFP1-10 | ✓ | 2.4E+09 | 208.9 |

TABLE 2
Exemplary gectosome incorporating unique human fusiogenic protein.

| Human endogenous | | GFP signal | Particle | Particle |
|---|---|---|---|---|
| fusogenic proteins | Gectosomes Configuration | in 293T | concentration | size |
| Syncytin 1 | Syncytin1-GFP11/BlaM-Vpr-GFP1-10 | ✓ | 8 × 10E+7 | 82 |
| Syncytin 2 | Syncytin1-GFP11/BlaM-Vpr-GFP1-10 | ✓ | 1 × 10E+8 | 40 |
| HERV-K Env | HERV-K-GFP11/BlaM-Vpr-GFP1-10 | ✓ | 6 × 10E+7 | 82 |

### REFERENCES

1. Ackaert, Chloé, Natalia Smiejkowska, Catarina Xavier, Yann G. J. Sterckx, Sofie Denies, Benoit Stijlemans, Yvon Elkrim, et al. 2021. “Immunogenicity Risk Profile of Nanobodies.” Frontiers in Immunology 12: 632687.
2. Cabantous, Stéphanie, Thomas C Terwilliger, and Geoffrey S Waldo. 2005. “Protein Tagging and Detection with Engineered Self-Assembling Fragments of Green Fluorescent Protein.” Nature Biotechnology 23 (1): 102-7.
3. Cabantous, Stéphanie, and Geoffrey S. Waldo. 2006. “In Vivo and in Vitro Protein Solubility Assays Using Split GFP.” Nature Methods 3 (10): 845-54.
4. Clackson, T., W. Yang, L. W. Rozamus, M. Hatada, J. F. Amara, C. T. Rollins, L. F. Stevenson, et al. 1998. “Redesigning an FKBP-Ligand Interface to Generate Chemical Dimerizers with Novel Specificity.” Proceedings of the National Academy of Sciences of the United States of America 95 (18): 10437-42.
5. Crabtree, G. R., and S. L. Schreiber. 1996. “Three-Part Inventions: Intracellular Signaling and Induced Proximity.” Trends in Biochemical Sciences 21 (11): 418-22.
6. Jin, L., H. Zeng, S. Chien, K. G. Otto, R. E. Richard, D. W. Emery, and C. A. Blau. 2000. “In Vivo Selection Using a Cell-Growth Switch.” Nature Genetics 26 (1): 64-66.
7. Kang, Shoukai, Kristian Davidsen, Luis Gomez-Castillo, Huayi Jiang, Xiaonan Fu, Zengpeng Li, Yu Liang, et al. 2019. “COMBINES-CID: An Efficient Method for De Novo Engineering of Highly Specific Chemically Induced Protein Dimerization Systems.” Journal of the American Chemical Society 141 (28): 10948-52.
8. LaFleur, Martin W., Thao H. Nguyen, Matthew A. Coxe, Kathleen B. Yates, Justin D. Trombley, Sarah A. Weiss, Flavian D. Brown, et al. 2019. “A CRISPR-Cas9 Delivery System for in Vivo Screening of Genes in the Immune System.” Nature Communications 10 (1): 1668.
9. Mangeot, Philippe-Emmanuel, Sandra Dollet, Mathilde Girard, Claire Ciancia, Stéphane Joly, Marc Peschanski, and Vincent Lotteau. 2011. “Protein Transfer Into Human Cells by VSV-G-Induced Nanovesicles.” Molecular Therapy 19 (9): 1656-66.
10. Mathieu, Mathilde, Lorena Martin-Jaular, Grégory Lavieu, and Clotilde Théry. 2019. “Specificities of Secretion and Uptake of Exosomes and Other Extracellular Vesicles for Cell-to-Cell Communication.” Nature Cell Biology 21 (1): 9-17.
11. Muthuswamy, S. K., M. Gilman, and J. S. Brugge. 1999. “Controlled Dimerization of ErbB Receptors Provides Evidence for Differential Signaling by Homo- and Heterodimers.” Molecular and Cellular Biology 19 (10): 6845-57.
12. Zhang, Xiaojuan, Quanbin Xu, Zhike Zi, Zeyu Liu, Chun Wan, Lauren Crisman, Jingshi Shen, and Xuedong Liu. 2020. “Programmable Extracellular Vesicles for Macromolecule Delivery and Genome Modifications.” Developmental Cell 55 (6): 784-801.e9.

1. A composition for delivery of a target molecule comprising:
a first fusion peptide having a membrane-fusion moiety and a first component of a split complement system;
a second fusion peptide having a second component of the split complement system and a first dimerization domain;
a third fusion peptide having a second dimerization domain and a target molecule;
wherein said membrane-fusion moiety is anchored to a cell membrane of a cell that forms an extracellular vesicle (EV); and
wherein the first, second, and third fusion peptides form a trimeric complex thereby loading said target molecule into said EV forming a gectosome for delivery to a target cell.

2. The composition of claim 1, wherein said split complement system is selected from: a split GFP system, a NanoBiT split ubiquitin system, a split beta-gal system, a split luciferase system, a split mCherry system, a split FRET system, and a split biotin system.

3. The composition of claim 1, wherein said target molecule is selected from: a ribonucleoprotein (RNP) complex, a protein, a protein fragment, a therapeutic protein, a protein-nucleotide complex, a cellular reprogramming protein, a labeled protein, a peptide aptamer, an antibody, an antibody fragment, tumor specific antigen peptide, a genome editing enzyme, an antigen, an oligonucleotide, a meganuclease, a nucleic acid, a DNA molecule, an RNA molecule, an RNAi molecule, a single guide RNA, a protein involved in the RNA-induced silencing complex (RISC), a therapeutic compound, a nanoparticle, a ligand, a prodrug, a nuclease, L7Ae, Ago2, Cas9, dCas9, SaCas9, dSaCas9, LwaCas13, RfxCas13d, Cas13, C2c1, C2C3, C2c2, Cfp1, MAD7, CasX, CRISPR crRNA, base editors constructed by dCas9 fusion to a cytidine deaminase protein, CRISPRi, CRISPRa, CRISPRX, CRISPR-STOP, a TALEN nuclease, a Zinc-Finger nuclease, a CRE recombinase, a catalase, and BDNF.

4. The composition of claim 1, wherein said membrane-fusion moiety is selected from: a cell or tissue-specific membrane-fusion moiety, a viral membrane-fusion moiety, or a human membrane-fusion moiety.

5. The composition of claim 4, wherein said cell or tissue-specific membrane-fusion moiety is selected from: one or more Vesicular stomatitis variants G fusion proteins (VSV-G or VSV-G-NJ), a Carajas virus G fusion protein (CARA-G), a Chandipura vesiculovirus G fusion protein (CNV-G), a SARS-CoV2 S protein (S), a MERS-CoV S protein (M), a Cocal virus G fusion protein (COCA-G), a Maraba virus G fusion protein (MARA-G), an Ebola virus G fusion protein (MOKV-G), a rabies virus (RAB-G) G fusion protein, a Nipah virus G fusion protein (NIV-G), a Chikungunya virus G fusion protein (CHIKV-G), a Syncytin 1 G fusion protein (SYN1-G), a Syncytin 2 G fusion protein (SYN2-G), and a HERV-K G fusion protein (HERVK-G).

6. The composition of claim 4, wherein said cell or tissue-specific membrane-fusion moiety is selected from:
a CNV-G protein according to SEQ ID NO.'s 1 or 2, or a fragment or variant thereof;
a Cara-G protein according to SEQ ID NO.'s 4-6, or a fragment or variant thereof;
a SARS-G protein according to SEQ ID NO. 7, or a fragment or variant thereof;
a VSV-G protein according to SEQ ID NO.'s 10-11, 13 or a fragment or variant thereof;
a RAB-G protein according to SEQ ID NO. 14, or a fragment or variant thereof;
a NIV-G protein according to SEQ ID NO.'s 15 or 16, or a fragment or variant thereof;
a COCA-G protein according to SEQ ID NO. 17, or a fragment or variant thereof;
a MARA-G protein according to SEQ ID NO. 18, or a fragment or variant thereof;
a MOKV-G protein according to SEQ ID NO. 19, or a fragment or variant thereof;
a CHIKV-G protein according to SEQ ID NO. 20, or a fragment or variant thereof;
a MERS-G protein according to SEQ ID NO. 21, or a fragment or variant thereof;
a SYN1-G protein according to SEQ ID NO. 22, or a fragment or variant thereof;
a SYN2-G protein according to SEQ ID NO. 23, or a fragment or variant thereof; and
a HERVK-G protein according to SEQ ID NO. 24, or a fragment or variant thereof.

7. The composition of claim 1, wherein said first dimerization domain and said second dimerization domain form a heterodimer directly, or through a dimerization mediator, wherein said dimerization mediator further comprises AP21967 or AP20187.

8. The composition of claim 1, said first dimerization domain comprises a DmrC domain and said second dimerization domain comprises a DmrA domain.

9. The composition of claim 1, wherein said first dimerization domain and said second dimerization domains are nanobodies, and wherein said first dimerization domain is nanobody CA14, and the second dimerization domain is DB21.

10. The system of claim 9, wherein cannabidiol (CBD) is contacted with the cell and induces gectosome production.

11. The composition of claim 1, wherein one or more of the fusion peptides includes a protein moiety that increases delivery of said target molecule, wherein said motif is selected from: a Gag peptide motif, and a p6^{Gag} peptide according to SEQ ID NO. 2.

12. (canceled)

13. The composition of claim 1, wherein the cell comprises a packaging cell.

14. The composition of claim 13, wherein endogenous expression of Munc13-4 in said packaging cell is disrupted.

15. The composition of claim 1, wherein said target molecule comprises a peptide moiety configured to bind a target oligonucleotide, wherein said target oligonucleotide is further selected from the group consisting of: an RNA molecule; an siRNA molecule, an RNAi molecule; and a single guide RNA with a CRISPR crRNA.

16. The composition of claim 15, wherein said peptide moiety comprises a peptide moiety selected from the group consisting of: a MS2-coat protein, a Ago2 peptide, LwaCas13a, and RfxCas13d, peptide.

17-45. (canceled)

46. A non-immunogenic composition for delivery of a target molecule comprising:
a first fusion peptide having a membrane-fusion moiety and a first component of a split complement system;
a second fusion peptide having a second component of the split complement system and a first dimerization domain;
a third fusion peptide having a second dimerization domain and a target molecule;
wherein said membrane-fusion moiety is configured to be anchored to a cell membrane of a cell that forms an extracellular vesicle (EV);
wherein the first, second, and third fusion peptides form a trimeric complex thereby loading said target molecule into said EV forming a gectosome for delivery to a target cell; and
wherein said split component system is a split Vex-GFP system, and wherein said first dimerization domain and said second dimerization domains are nanobodies, and wherein said first dimerization domain is nanobody CA14, and said second dimerization domain is DB21.

47. The composition of claim 46, wherein cannabidiol (CBD) is contacted with the cell and induces gectosome production.

48. The composition of claim 46, and wherein said gectosome displays a CD47 peptide.

49. The composition of claim 46, wherein said target molecule is selected from: a ribonucleoprotein (RNP) complex, a protein, a protein fragment, a therapeutic protein, a protein-nucleotide complex, a cellular reprogramming protein, a labeled protein, a peptide aptamer, an antibody, an antibody fragment, tumor specific antigen peptide, a genome editing enzyme, an antigen, an oligonucleotide, a meganuclease, a nucleic acid, a DNA molecule, an RNA molecule, an RNAi molecule, a single guide RNA, a protein involved in the RNA-induced silencing complex (RISC), a therapeutic compound, a nanoparticle, a ligand, a prodrug, a nuclease, L7Ae, Ago2, Cas9, dCas9, SaCas9, dSaCas9, LwaCas13, RfxCas13d, Cas13, C2c1, C2C3, C2c2, Cfp1, MAD7, CasX, CRISPR crRNA, base editors constructed by dCas9 fusion to a cytidine deaminase protein, CRISPRi, CRISPRa, CRISPRX, CRISPR-STOP, a TALEN nuclease, a Zinc-Finger nuclease, a CRE recombinase, a catalase, and BDNF.

50. The composition of claim 1, wherein said gectosome displays a CD47 peptide.
